(19) United States
(12) Patent Application Publication
(10) Pub. No.: US 2004/0230458 A1
(43) Pub. Date: Nov. 18, 2004

(54) Cyber hospital system for providing doctors' assistances from remote sites

(75) Inventors: Takuzo Takayama, Tochigi-ken (JP); Yoichi Takada, Tochigi-ken (JP); Keisuke Hashimoto, Tochigi-ken (JP); Hiroshi Asahina, Tochigi-ken (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 10/784,279

(22) Filed: Feb. 24, 2004

(30) Foreign Application Priority Data
Feb. 26, 2003 (JP) 2003-049055
Feb. 28, 2003 (JP) 2003-053192

(51) Int. Cl. G06F 17/60; G06F 7/00; G06F 17/00; A61B 5/00

(52) U.S. Cl. 705/3; 707/104.1

(57) ABSTRACT

A cyber hospital system is connected to a remote terminal through a network. The cyber hospital system includes an input unit, a first processor, and a transmitter. The input unit is configured to input patient condition information from the remote terminal through the network. The first processor is configured to collect first doctor information based on the patient condition information. The transmitter is configured to transmit the first doctor information to the remote terminal.

### CROSS-REFERENCE TO RELATED APPLICATION

This application is based upon and claims the benefit of priority from prior Japanese Patent Applications No. P2003-49055, filed on Feb. 26, 2003 and No. P2003-53192, filed on Feb. 28, 2003, the entire contents of which are incorporated herein by reference.

### BACKGROUND OF THE INVENTION

1. Field of the Invention

The present invention relates to a cyber hospital system enabling communications among a patient, one or more doctors and medical examination facilities located in different palaces, respectively. The present invention further relates to a medical information supply system which collects patient information and is used independently from or included in the cyber hospital system.

2. Discussion of the Background

Usually each person has his or her family doctor in a clinic. When the clinic does not have an equipment on a facility for implementing a necessary examination, the family doctor requests another hospital to implement the examination. In the above case, once the doctor has made the request, the doctor and the patient typically leave the rest to the hospital. Therefore, the doctor and the patient cannot choose, for example, a facility to be used for the examination, a doctor who interprets a result of the examination and prepares an examination report, or a doctor who makes a diagnosis.

Also, when the patient is referred to a hospital the patient goes to the hospital and is diagnosed by a doctor who works in the hospital and is treated by the same or another doctor who also works in the hospital. If, however, the patient prefers to receive a diagnosis and a treatment from a certain doctor in a hospital located far from the patient, the patient has to go to that hospital. Otherwise, the patient cannot be diagnosed and treated by the doctor.

Meanwhile, in an emergency case or when a patient is unconscious, it is difficult to find someone who knows a health or medical condition of the patient. Furthermore, it is generally important for a doctor to know as much information about the patient as possible to perform a medical examination, make a diagnosis, and treat the patient. It is, however, not easy to obtain all the medical information about a patient since a patient may usually go to several hospitals as well as his or her family doctor.

### SUMMARY OF THE INVENTION

According to a first aspect of the present invention, there is provided a cyber hospital system connected to a remote terminal through a network. The cyber hospital system includes an input unit, a first processor, and a transmitter. The input unit is configured to input patient condition information from the remote terminal through the network. The first processor is configured to collect first doctor information based on the patient condition information. The transmitter is configured to transmit the first doctor information to the remote terminal.

According to a second aspect of the present invention, there is provided a cyber hospital system connected to a remote terminal through a network. The system includes an input unit, a processor, and a transmitter. The input unit is configured to input patient location information from the remote terminal through the network. The processor is configured to collect medical facility information based on the patient location information. The transmitter is configured to transmit the medical facility information to the remote terminal.

According to a third aspect of the present invention, there is provided a medical information supply system connected to a remote terminal and a plurality of databases. The system includes an input unit, a processor, and a transmitter. The input unit is configured to input first patient information and use information from the remote terminal. The processor is configured to make a request to one or more of the databases so as to collect second patient information based on the first patient information and the use information. The transmitter is configured to transmit the second patient information to the remote terminal.

According to a fourth aspect of the present invention, there is provided a method of supplying a remote terminal with certain information. The method begins by inputting first patient information and use information from the remote terminal. The method continues by making a request to one or more databases so as to collect second patient information based on the first patient information and the use information. The method further continues by transmitting the second patient information to the remote terminal as the certain information.

According to a fifth aspect of the present invention, there is provided a method of medical information processing. The method begins by inputting patient condition information and patient identification information from a remote terminal through a network. The method continues by collecting patient information based on the patient identification information, and deducing a medical condition of the patient based on the patient condition information and the patient information. The method further continues by preparing a medical action plan based on the deduced medical condition, and forecasting a future condition of the patient which is expected by implementing the medical action plan on the patient.

### BRIEF DESCRIPTION OF THE DRAWINGS

A more complete appreciation of embodiments of the present invention and many of its attendant advantages will be readily obtained by reference to the following detailed description considered in connection with the accompanying drawings, in which:

FIG. 1 is an illustration showing an exemplary aspect of a cyber hospital system according to a first embodiment of the present invention;

FIG. 2 is a block diagram showing an exemplary cyber hospital system connected to hospitals and others according to the first embodiment of the present invention;

FIG. 3 is a flowchart showing a first part of an exemplary flow of processes for and after establishing a cyber hospital according to the first embodiment of the present invention;

FIG. 4 is a flowchart showing a second part of the exemplary flow of processes for and after establishing the cyber hospital according to the first embodiment of the present invention;

FIG. 5 is a flowchart showing a third part of the exemplary flow of processes for and after establishing the cyber hospital according to the first embodiment of the present invention;

FIG. 6 is an illustration showing a first exemplary schema of a medical information supply system according to a second embodiment of the present invention;

FIG. 7 is an illustration showing a second exemplary schema of the medical information supply system according to the second embodiment of the present invention;

FIG. 8 is an illustration showing a third exemplary schema of the medical information supply system according to the second embodiment of the present invention;

FIG. 9 is an illustration showing a first example of display window displayed in a user's terminal according to the second embodiment of the present invention;

FIG. 10 is an illustration showing a second example of display window displayed in a user's terminal according to the second embodiment of the present invention;

FIG. 11 is an illustration showing an example of a processing flow in a virtual patient agent engine according to the second embodiment of the present invention;

FIG. 12 is a flowchart showing an example of a first part of a detailed processing flow regarding the virtual patient agent engine according to the second embodiment of the present invention;

FIG. 13 is a flowchart showing an example of a second part of the detailed processing flow regarding the virtual patient agent engine according to the second embodiment of the present invention;

FIG. 14 is an illustration showing an example of a case that the medical information supply system is used independently in relationship with a plurality of databases and user's terminals according to a third embodiment of the present invention;

FIG. 15 is a block diagram showing an exemplary configuration of the medical information supply system according to the third embodiment of the present invention;

FIG. 16 is a flowchart showing a first part of an exemplary operation flow of the medical information supply system according to the third embodiment of the present invention;

FIG. 17 is a flowchart showing a second part of the exemplary operation flow of the medical information supply system according to the third embodiment of the present invention; and

FIG. 18 is a flowchart showing a third part of the exemplary operation flow of the medical information supply system according to the third embodiment of the present invention.

### DESCRIPTION OF THE PREFERRED EMBODIMENTS

Embodiments of the present invention will be described with reference to the accompanying drawings.

(First Embodiment)

A cyber hospital according to a first embodiment of the present invention is based on a concept in which any desired doctors and various medical examination facilities are available to a patient. The doctors may be located in different clinics, hospitals, and/or medical centers. The clinics may be ones located near the patient (i.e., where the patient lives or stays when the patient is outside the house). The hospitals or the medical centers maybe located anywhere in the world. The medical centers include, for example, a cancer center and a cardiovascular center. The doctors include, for example, a family doctor or a primary physician of the patient particularly when it is a clinic, diagnosticians, medical specialists, and image interpretation doctors. The medical examination facilities may include medical imaging equipments, medical examination equipments, and medical therapy equipments. The medical imaging equipments are, for example, an X-ray diagnosis apparatus, an X-ray computed tomography apparatus, and a magnetic resonance imaging apparatus. Further examples of the medical examination facilities will be described later.

The cyber hospital system includes a feature of providing communications among the patient and the doctors and a feature of supplying necessary information to the patient and the doctors.

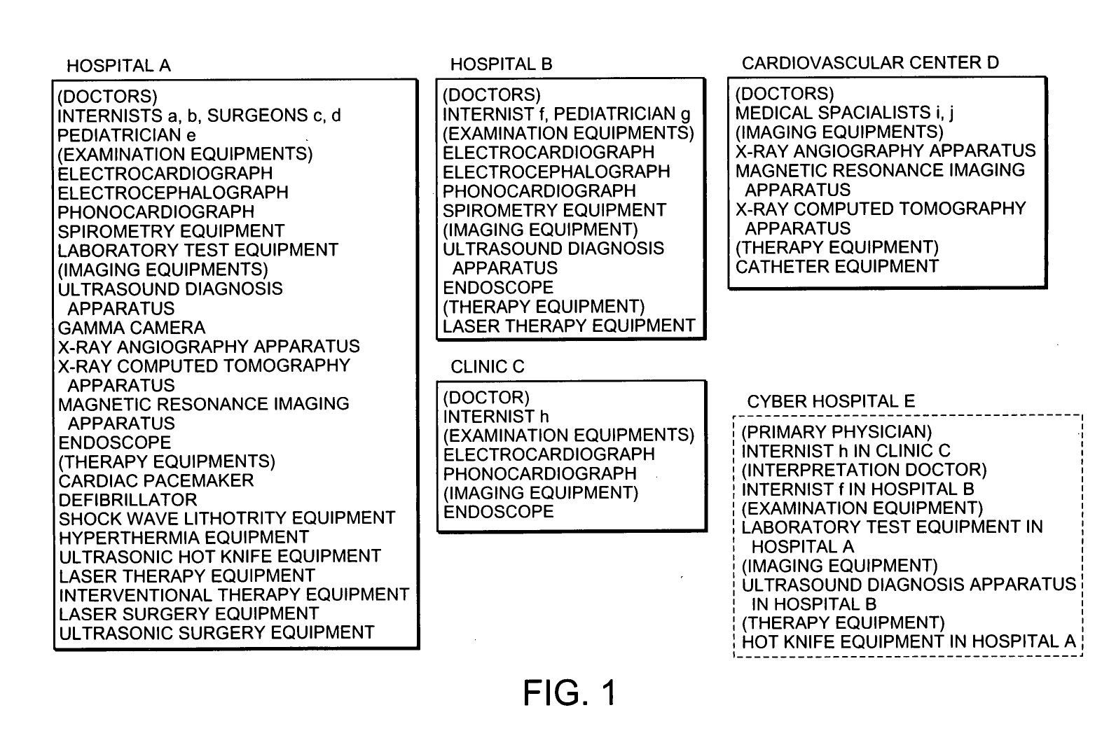

The cyber hospital system according to the first embodiment will be described in detail with reference to FIGS. 1 to 5.

FIG. 1 is an illustration showing an exemplary aspect of the cyber hospital system according to the first embodiment of the present invention. As shown in FIG. 1, there are real-existent hospitals A and B, a real-existent clinic C, and a real-existent cardiovascular center D, which may be located at remote places and away from each other.

<Hospital A>

In one example, internists a and b, surgeons c and d, and a pediatrician e may work in the hospital A. As medical examination equipments, there are an electrocardiograph, an electrocephalograph, a phonocardiograph, a spirometry equipment, and a laboratory test equipment. In addition, the hospital A may have an ultrasound diagnosis apparatus, a gamma camera (or a nuclear medicine diagnosis apparatus), a cardiovascular X-ray diagnosis apparatus such as an X-ray angiography apparatus, an X-ray computed tomography apparatus, a magnetic resonance imaging apparatus, and an endoscope. Further, as therapy equipments provided are a cardiac pacemaker, a defibrillator, a shock wave lithotrity equipment, a hyperthermia equipment, an ultrasonic hot knife equipment, a laser therapy equipment, an interventional therapy equipment, a laser surgery equipment, and an ultrasonic surgery equipment.

<Hospital B>

An internist f and a pediatrician g may work in the hospital B. As medical examination equipments, there may be an electrocardiograph, an electrocephalograph, a phonocardiograph, and a spirometry equipment. In addition, the hospital B may have an ultrasound diagnosis apparatus and an endoscope. Further, a laser therapy equipment is provided as a therapy equipment.

<Clinic C>

An internist h may work for the clinic C. An electrocardiograph and a phonocardiograph are provided as medical examination equipments. The clinic C has, for example, an endoscope.

<Cardiovascular Center D>

Medical specialists i and j may work in the cardiovascular center D. As medical imaging equipments, there are a cardiovascular X-ray diagnosis apparatus, a magnetic resonance imaging apparatus, and an X-ray computed tomography apparatus. In addition, the cardiovascular center D has a catheter equipment as a therapy equipment.

<Cyber Hospital E>

While the hospitals A and B, the clinic C, and the cardiovascular center D exist as described above, a cyber hospital is formed for a particular patient and may have a team including, for example, the internist h in the clinic C as a primary physician, and the internist f in the hospital B as an image interpretation doctor. In the cyber hospital E, the laboratory test equipment provided in the hospital A, the ultrasound diagnosis apparatus provided in the hospital B, and the ultrasonic hot knife equipment provided in the hospital A are used for an examination, an imaging, and a therapy as a part of the team.

The patient goes to hospital A for the laboratory test and the therapy. The patient also goes to hospital B for the ultrasound imaging. The internists f and h communicate with each other through a network connecting the hospitals B and C. The patient may join the communication between the internists f and h. A detailed aspect of the communication among them will be described later.

Although one example of the cyber hospital has been described according to the first embodiment, a cyber hospital according to the invention can be formed by the selection of any doctors and any medical examination facilities from a plurality of hospitals, a plurality of medical centers, a plurality of clinics, and a plurality of medical institutes. In other words, a patient can select one or more desired doctors in the world for his or her medical problem. Further, the patient or the doctors selected by the patient may select one or more desired medical examination facilities such as medical imaging, examination, and/or therapy equipments provided in different hospitals. The hospitals, the medical centers, the clinics, and the medical institutes may be located anywhere domestically or in the world.

A cyber hospital system according to the first embodiment includes an information collection feature, a database feature, and a communication feature. According to the information collection feature, the cyber hospital system collects patient information such as patient medical information and patient general information. The cyber hospital system also collects information regarding a latest guideline for diagnoses and therapies such as, for example, ‘Evidence Based Medicine’ (EBM). The above information maybe collected from databases provided in a plurality of hospitals. According to the database feature, the cyber hospital system stores or archives information which is necessary to support the selection of the doctors and the medical examination facilities. Such archived information includes, for example, doctors' names in a plurality of hospitals and clinics, how to access to the doctors, and thedoctors' fields of specialization. The archived information further includes medical specialists' names all over the world, the medical specialists' fields of specialization, the number of experienced operations, techniques, mortality, names of hospitals for which they work, and performances (achievements). According to the communication feature, the cyber hospital system can communicate with a patient terminal provided in the house of the patient and hardware, such as patient terminals or communication terminals and medical examination facilities, provided in hospitals or medical centers all over the world through one or more types of electronic networks. Further, the communication feature may provide a communication hotline by, for example, an IP (Internet Protocol) telephone between the patient and the doctor in the world. Accordingly, the patient can directly communicate with the doctor in real time.

A term ‘database’ may also include a meaning of a server, a memory, or any other storage in embodiments of the present invention.

In order to realize the cyber hospital system mentioned above, the following supplemental or support features may be implemented.

A feature that the patient can decide one or more doctors who can access to an information system storing all or most of the patient information as if the information system represented (or acted as) the patient (or an alter ego of the patient)

The information system may not necessarily store the above mentioned information itself. Alternatively, the information system may have a feature of accessing to a plurality of databases provided in other hospitals so as to acquire necessary information.

In those cases, such an information system may be called a virtual patient system. Details of the virtual patient system will be described later.

A feature for preparing an information system which stores all or most of doctors' (or medical specialists’) names, the doctors' fields of specialization, the number of experienced operations, techniques, mortality, names of hospitals for which they work, and performances (achievements)

A feature for limitedly allowing a particular doctor to answer all the medical questions from the patient

A feature for allowing any doctor to request a medical examination to any hospital and to receive a result of the examination

A feature for allowing a doctor to designate apparatuses or equipments to be used in a medical examination when the doctor requests the medical examination

A feature of allowing a doctor to designate who should determine or interpret a result of a medical examination when the doctor requests the medical examination

A feature for showing performances or accuracy of medical examinations, image interpretation, determination or judgment, and the like with regard to every doctor, apparatus, and equipment

A feature for presenting a latest technique of medical examination or therapy based on a symptom

Turning now to requests to the cyber hospital system by a patient, a primary physician, and a medical specialist, the requests may be assumed as follows.

<Requests by Patient>

(Primary Physician Selection)

The patient may want to decide a primary physician at his/her will by referring to the doctor information. The primary physician may become familiar with the patient health condition.

The patient may want to change a current primary physician to others whenever the patient does not like the current primary physician.

The patient may want to allow a primary physician to disclose information of the patient in the virtual patient system to other doctors who perform a medical examination, a diagnosis, or a therapy after they have obtained a permission of the patient.

(Medical Specialists Selection)

The patient may want to know medical specialists' names, the medical specialists' fields of specialization, the number of experienced operations, techniques, mortality, names of hospitals for which they work, and performances (achievements).

The patient may want a desired medical specialist to diagnose and treat him/her even remotely.

(Medical Examination and Therapy Selection)

The patient may want to designate apparatuses and equipments to be used in medical examinations and therapy.

The patient may want to designate a doctor who interprets a result of a medical examination and a therapy and prepares a medical report based on the interpretation.

The patient may want to know performances or accuracy of medical examinations, image interpretation, determination or judgment, and the like with regard to every doctor, apparatus, and equipment.

(When the Patient Feels to Lose Health)

All or most of basic information of the patient in the virtual patient system is usually automatically updated and automatically sent to a primary physician. Before the patient gets ill, the primary physician warns the patient. In addition, a precaution and/or a medical treatment plan are presented so as to prevent the patient from getting worse.

The automatic update means, for example, all the apparatuses and equipments automatically transfer data obtained by examinations, therapies, and any other performances to the virtual patient system. Another example is that the apparatuses and equipments automatically inform the virtual patient system about the examinations, the therapies, and other performances so that the virtual patient system can update information of the patient. The doctors or the patient can also input necessary information into the virtual patient system.

(In Case of Emergency)

All or most of the patient information of the patient in the virtual patient system is transmitted to an ambulance or a hospital which the patient is brought into. Alternatively, location information of the patient information maybe transmitted to the ambulance or the hospital.

This transmission is helpful to apply an appropriate treatment to the patient when the patient cannot speak or cannot properly answer doctor's questions.

<Requests by Primary Physician>

(Regarding the Patient)

The primary physician may want to obtain information of the patient in the virtual patient system.

(Medical Specialists Selection)

The primary physician may want to know medical specialists' names, the medical specialists' fields of specialization, the number of experienced operations, techniques, mortality, names of hospitals for which they work, and performances (achievements).

The primary physician may want to introduce the patient to a medical specialist and wants the medical specialist to cure the patient.

<Requests by Medical specialist>

The medical specialist may want to have or access an information system, which stores a latest guideline for diagnoses and therapies such as the EBM, so as to always apply a latest medical treatment to the patient.

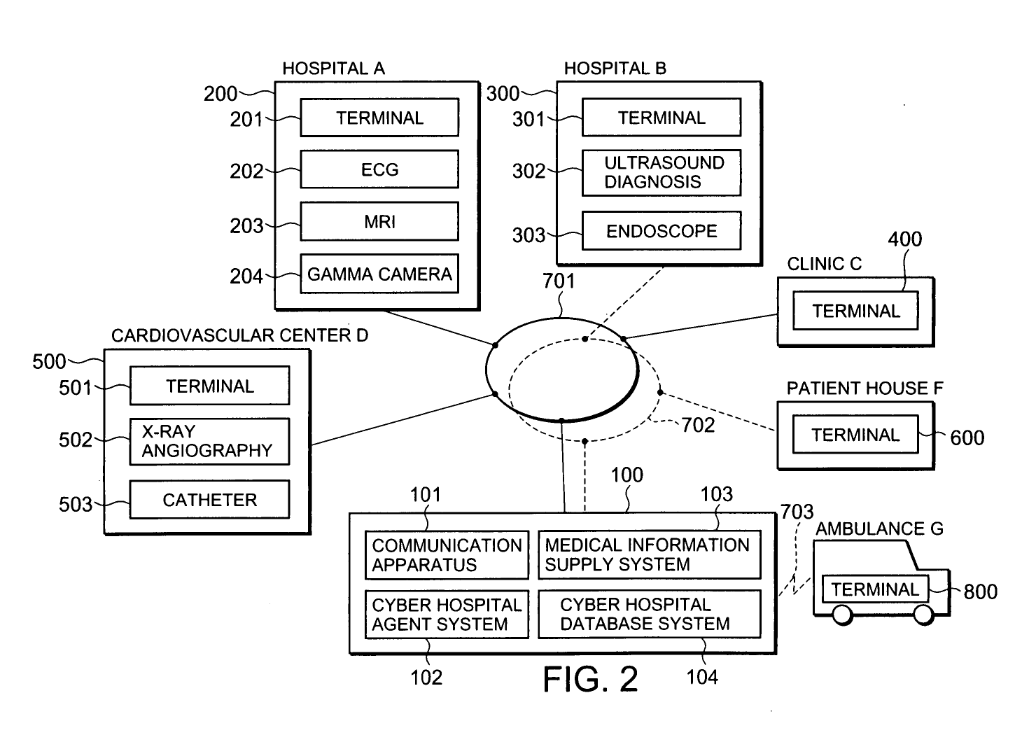

FIG. 2 is a block diagram showing an exemplary cyber hospital system connected to real-existent hospitals and others according to the first embodiment of the present invention.

As shown in FIG. 2, a cyber hospital system 100 is connected to an information system 200 provided in a hospital A, a consultation room terminal 400 provided in a clinic C, and an information system 500 provided in a cardiovascular center D through a network 701. The network 701 may be the Internet, the public telephone network, the optical fiber communication network, or any other possible communication means. The cyber hospital system 100 is further connected to an information system 300 provided in a hospital B and to a patient terminal 600 provided in a patient's house F through a network 702. The network 701 may be the Internet, the public telephone network, the optical fiber communication network, or any other possible communication means. The cyber hospital system 100 may be further connected to a mobile terminal 800 provided in an ambulance G through a radio network 703. Any terminal described in embodiments of the present invention may include an input feature and a display feature. An example of one terminal is a (personal) computer terminal. Also, a terminal used for other purpose, such as, for example, an image display, a report display, an accountant, and/or hospital information can be used as the terminal used in embodiments of the present invention.

The hospital A may be functioned as a central hospital. The hospital B may be functioned as a local hospital. The clinic C may be located near the patient house F. The cardiovascular center D may be functioned as a medical center. The mobile terminal 800 may be either fixed to the ambulance G or portable from the ambulance G.

The information system 200 provided in the hospital A may include an ordering system, an accounting system, and a picture archiving and communication system (PACS). For example, the information system 200 may include a consultation room terminal 201, an electrocardiograph (ECG) 202, a magnetic resonance imaging apparatus (MRI) 203, and a gamma camera 204. The consultation room terminal 201 is provided in a consultation room in the hospital A. There may be a plurality of consultation room terminals 201. The electrocardiograph 202 is used as a medical examination equipment. The magnetic resonance imaging apparatus 203 and the gamma camera are used as medical imaging equipments.

The information system 300 provided in the hospital B may include an ordering system, an accounting system, and a PACS. For example, the information system 300 includes a consultation room terminal 301, an ultrasound diagnosis apparatus 302, and an endoscope 303. The consultation room terminal 301 is provided in a consultation room in the hospital B. There may be a plurality of consultation room terminals 301. The ultrasound diagnosis apparatus 302 and the endoscope 303 are used as medical imaging equipments.

The information system 500 provided in the cardiovascular center D may include an ordering system, an accounting system, and a PACS. For example, the information system 500 includes a consultation room terminal 501, a cardiovascular X-ray diagnosis apparatus 502 such as an X-ray angiography apparatus, and a catheter equipment 503. The consultation room terminal 501 is provided in a consultation room in the cardiovascular center D. There may be a plurality of consultation room terminals 501. The cardiovascular X-ray diagnosis apparatus 502 is used as a medical imaging equipment. The catheter equipment 503 is used as a therapy equipment.

The cyber hospital system 100 includes a communication apparatus 101, a cyber hospital agent system 102, a medical information supply system 103, and a cyber hospital database system 104.

The communication apparatus 101 includes various communication functions and is connected to the networks 701, 702, and 703.

The cyber hospital agent system 102 receives a request for establishing a cyber hospital from a patient, a doctor, or an ambulance crew through the patient terminal 600, the consultation room terminal 201, 301, 400, or 501, or the mobile terminal 800. Based on the request, the cyber hospital agent system 102 makes the cyber hospital database system 104 to transmit doctor information, medical specialist information, interpretation doctor information, and/or medical examination facility information which respond to or correspond to patient information included in the request. The information transmission is made to the terminal which made the request (a request origination terminal). The cyber hospital agent system 102 then receives selection information regarding a primary physician, a medical specialist, an interpretation doctor, and a medical facility from the request origination terminal. The cyber hospital agent system 102 determines a doctor belonging to one of the hospitals A and B, and the clinic C as a primary physician in accordance with the selection information regarding the primary physician. The cyber hospital agent system 102 also determines a doctor belonging to the cardiovascular center D as a medical specialist in accordance with the selection information regarding the medical specialist. The cyber hospital agent system 102 further determines a doctor belonging to one of the hospitals A and B as an interpretation doctor in accordance with the selection information regarding the interpretation doctor. Also, the cyber hospital agent system 102 may determine one or more medical examination facilities belonging to at least one of the hospitals A and B, and the cardiovascular center D in accordance with the selection information regarding the medical examination facility. Accordingly, a cyber hospital including the determined primary physician, medical specialist, interpretation doctor, (the ambulance crew,) and the determined medical examination facilities is established for the patient. The determined primary physician, medical specialist, interpretation doctor, (the patient, the ambulance crew, the requesting doctor,) and the determined medical examination facilities are communicable to one another through one or more of the networks 701 to 703.

The medical information supply system 103 may request and collect necessary information from databases provided in a plurality of hospitals through the communication apparatus 101, for example. The necessary information may include patient information and information regarding a latest guideline for diagnoses and treatments such as, for example, the EBM. The medical information supply system 103 includes the virtual patient system for collecting the patient information as a part of the medical information supply system 103. Alternatively, the medical information supply system 103 may not include but be connected to the virtual patient system. The virtual patient system can be replaced with any other possible feature of collecting the patient information in application for the medical information supply system 103.

The medical information supply system 103 also includes another feature of supplying the request origination terminal and other necessary terminal(s) with the collected information and other information prepared by the virtual patient system based on the collected information. Details of the medical information supply system 103 will be described later.

The cyber hospital database system 104 includes one or more databases which store information of doctors, medical specialists, interpretation doctors, medical examination equipments, medical imaging equipments, and therapy equipments with regard to a plurality of medical facilities including hospitals, medical centers, and clinics. For example, such databases store information helpful to select a primary physician, doctors, medical specialists, and medical examination facilities. For the selection of the primary physician, the doctors, and the medical specialists, the databases may store information of doctors' names in a plurality of hospitals and clinics, how to access to the doctors, the doctors' fields of specialization, names of medical specialists all over the world, the medical specialists' fields of specialization, the number of experienced operations, techniques, mortality, names of hospitals for which they work, and performances (achievements). The various types of information mentioned above may be stored in external databases in part.

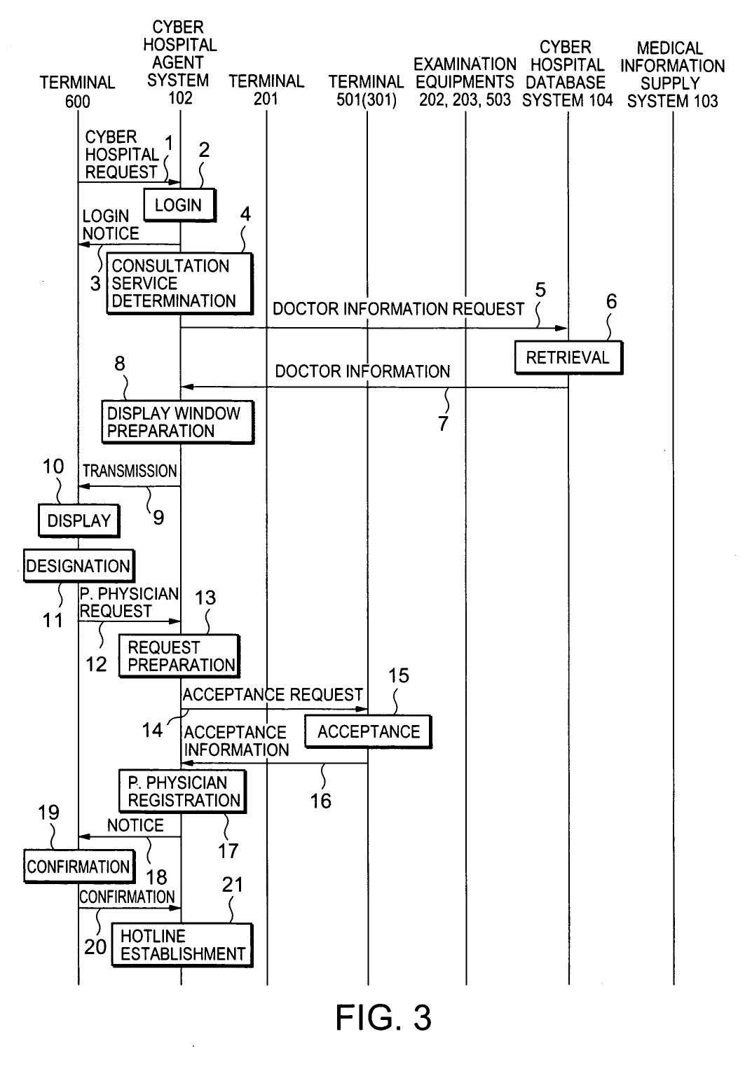

FIGS. 3 to 5 are flowcharts showing an exemplary operation flow for and after establishing a cyber hospital according to the first embodiment of the present invention. Here, the operation is described by taking an example of a case that a patient makes a request for establishing a cyber hospital. A request for establishing a cyber hospital is not limited to the case by the patient, but may also be made by an ambulance crew, a doctor in a hospital or a clinic, a medical specialist, or any other permitted person. In FIGS. 3 to 5, the operation is mainly described about the cyber hospital agent system 102.

As shown in FIG. 3, a patient operates the patient terminal 600 and inputs a user identification information (hereinafter referred to as a user ID) so as to make a request for establishing a cyber hospital (step 1). The establishment request also includes patient information of, for example, a symptom, a sex, an age, a body build, a life style, and an anamnesis. The cyber hospital agent system 102 authenticates the patient according to the user ID and permits the patient to login the cyber hospital system (step 2). Such permission is notified to the patient terminal 600 with a notice that an establishment request entry has been completed (step 3).

Also in the cyber hospital agent system 102, a suspected disease name is presumed based on the patient information included in the establishment request. In addition, it is determined in which service (or a consultation service) the patient should preferably be consulted. Such a determination may be executed based on the patient information included in the establishment request by a neuro computer (step 4).

Upon the presumption and the determination, the cyber hospital agent system 102 sends a doctor information request to the cyber hospital database system 104 (step 5). The doctor information request includes information of a presumed disease name and the determined consultation service. In some cases, the doctor information request may include the patient information included in the establishment request. The cyber hospital database system 104 retrieves doctor information based on the presumed disease name and the determined consultation service as keywords. In some cases, the retrieval may also be made based on the patient information included in the establishment request, in addition to the above-mentioned keywords (step 6). Retrieved doctor information regarding a plurality of doctors is sent to the cyber hospital agent system 102 from the cyber hospital database system 104 (step 7). The plurality of doctors are candidates to become a primary physician of the patient. The doctor information regarding each doctor may include a doctor's name, a personal history, a name of a hospital or a clinic for which the doctor works, an address of the hospital or the clinic, a field of specialization, an experience in diagnoses and therapy, and the like.

The cyber hospital agent system 102 prepares a display window having the doctor information in a form of a graphic user interface (GUI) (step 8). Data of the prepared GUI display window are transmitted to the patient terminal 600 through the communication apparatus 101 (step 9).

In response to the transmission by the cyber hospital agent system 102, the patient terminal 600 displays the GUI display window (step 10). The patient refers to the doctor information through the GUI display window and designates one desired doctor as his/her primary physician from among the plurality of primary physician candidates (i.e., doctors) (step 11). In response to this designation, a primary physician designation request is transmitted from the patient terminal 600 to the cyber hospital agent system 102 (step 12).

The cyber hospital agent system 102 responds to the primary physician designation request and prepares an acceptance request (step 13). When the doctor designated as the primary physician is a doctor in the hospital A, the prepared acceptance request is transmitted to the consultation room terminal 201 used by the designated doctor (step 14). The acceptance request may include the presumed disease name and the patient information included in the establishment request.

The designated doctor in the hospital A receives the acceptance request in the consultation room terminal 201. When the designated doctor accepts the request to become a primary physician of the patient, the designated doctor clicks on an acceptance button in a GUI display window displayed in the consultation room terminal 201 (step 15). The acceptance information is transmitted to the cyber hospital agent system 102 (step 16). If the designated doctor refuses, the designated doctor clicks on a refusal button in the GUI display window. In this case, the patient is asked to select other doctor.

When the acceptance information is received through the communication apparatus 101, the cyber hospital agent system 102 registers the doctor in the hospital A as a primary physician of the patient into a cyber hospital team (step 17). The cyber hospital agent system 102 transmits a message to the patient terminal 600 (step 18). The message notifies the patient of the acceptance and reads, for example, ‘your designated doctor has accepted to become your primary physician’. When the patient clicks on a confirmation button in the patient terminal 600 (step 19), the cyber hospital agent system 102 receives the confirmation (step 20) and establishes a communication hotline using, for example, an IP telephone between the consultation room terminal 201 of the primary physician and the patient terminal 600 of the patient (step 21). The cyber hospital agent system 102 may alternatively provide the consultation room terminal 201 and the patient terminal 600 with information necessary for establishing a similar communication hotline. In order to establish the communication hotline, the cyber hospital agent system 102 may be required a function similar to an exchange.

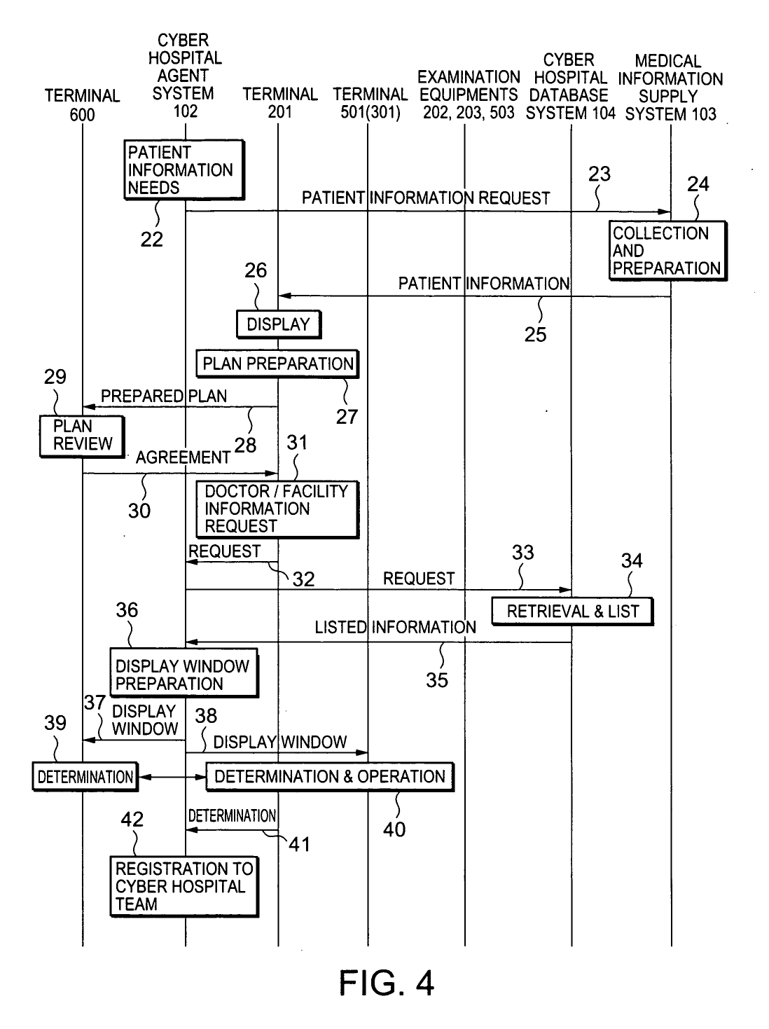

Turning now to FIG. 4, the cyber hospital agent system 102 needs to collect information of the patient (step 22). The cyber hospital agent system 102 sends a patient information supply request to the medical information supply system 103 (step 23). The patient information supply request may alternatively be transmitted directly from the consultation room terminal 201 to the medical information supply system 103. The patient information supply request may include patient information including, for example, a patient name, a patient ID, a sex, an age, and a symptom. In addition, the patient information supply request may also include user information including user role information and situation (or scene) information.

The user role information represents what the user is. In other words, the user role information indicates what person is going to use the medical information supply system 103 and access to information supplied by the medical information supply system 103. Possible users may be a doctor, a primary physician, a medical specialist, an ambulance crew, a patient, a nurse, and the like. Here, it is a case of a use by the primary physician. Therefore, the user role information is ‘primary physician’.

The situation information represents in what situation the user is going to use the medical information supply system 103 and access to information supplied by the medical information supply system 103. Possible situations may be an ordinary consultation, an ordinary image interpretation, an informed consent explanation, an emergency case, an emergency case in an ambulance (for remote diagnosis), and the like. Here, it is a case of an ordinary consultation. Therefore, the situation information is ‘ordinary consultation’.

The situation information may alternatively represent a place where the user is located at the time of using the medical information supply system 103 (or the cyber hospital). Such user location information (location information) is particularly useful to determine and present information of hospitals located near the user location. When the user needs only to know such hospital information, the medical information supply system 103 may allow the user to input only location information so as to supply the user with nearby hospitals information. Therefore, the user may not always have to input patient information such as patient ID and/or patient condition information as an input to the medical information supply system 103 in the above case.

On the contrary, there is a case that first of all, a patient wants a doctor to consult his/her symptom before considering various medical examinations. In such a case, the patient does not need to know the nearby hospitals information. When a doctor uses the medical information supply system 103, the doctor may not need to obtain the nearby hospital information, either. Therefore, when the user is the patient or the doctor in the above case, the medical information supply system 103 may allow the user not to input the location information as the situation information. In response, a cyber hospital is established without any medical examination facility. After the consultation, the user may be requested to input the location information as additional information so as to take medical examinations in a hospital near the user.

The medical information supply system 103 collects necessary information of the patient from databases provided in a plurality of hospitals, medical centers, and institutes through the communication apparatus 101. The information depends on the user (or user role information) and the situation (or situation information). That is, the necessary information is information which the user needs in the situation. When the medical information supply system 103 includes the virtual patient system, the virtual patient system practically collects the necessary information. If the medical information supply system 103 does not include the virtual patient system, the medical information supply system 103 accesses to the virtual patient system so as to collect information which the virtual patient system has collected.

The medical information supply system 103 deduces a disease name of the patient based on the patient symptom and the collected necessary information of the patient. Based on the deduced disease name, the medical information supply system 103 prepares a plurality of action plans as selectable candidates. Each action plan may include one or more of a diagnosis, a treatment, a care, a first aid in emergency, and the like which the user should carry out next. The medical information supply system 103 also collects information relating to the prepared action plans from the databases. Further, the medical information supply system 103 forecasts (or predicts), for example, a quality of life (QOL), an EBM, and mortality as a future expectation of the patient based on the collected information. The collected information for the forecast is not limited to the information mentioned above, but may also include all or some of the patient's symptom, sex, age, body build, life style, anamnesis, current hospital record, blood examination result, treatment, a type of medicine to be administered, a dose of the medicine, and a latest medical information.

Similar to the information collection case, when the medical information supply system 103 includes the virtual patient system, the virtual patient system practically implements the deduction, the preparation, and the forecast. If the medical information supply system 103 does not include the virtual patient system, the medical information supply system 103 accesses to the virtual patient system so as to collect results of the deduction, the preparation, and the forecast which the virtual patient system has implemented. Further, when the medical information supply system 103 does not include the virtual patient system, but has ability of the deduction, the preparation, and the forecast, the medical information supply system 103 only accesses to the virtual patient system so as to collect information which the virtual patient system has collected.

Turning back to FIG. 4, the medical information supply system 103 collects the information of the patient and prepares a display window in a form of a GUI based on the collected information of the patient, the deduction result, the prepared action plans, and the forecast result (step 24). The prepared display window including such information is transmitted to the consultation room terminal 201 in substantially or almost real time (step 25). The transmission may be implemented to the consultation room terminal 201 directly or via the cyber hospital agent system 102 from the medical information supply system 103.

In the consultation room terminal 201, the GUI display window is displayed (step 26). The primary physician refers to the displayed information, and studies and prepares an actual plan of a diagnosis and a therapy (step 27). The prepared plan is transmitted to the patient terminal 600 (step 28). The patient reviews the received plan (step 29). When the patient agrees to the plan, the primary physician is informed of the agreement from the patient (step 30).

The primary physician makes a doctor/facility information request for supplying information of, for example, biopsies, medical imaging examinations, therapies, and medical specialists, whichever are necessary to implement the plan (step 31). The doctor/facility information request is transmitted to the cyber hospital agent system 102 (step 32). The information of biopsies, medical imaging examinations, therapies, and medical specialists means information of hospitals, lists of doctors, interpretation doctors, and medical specialists, and details of those. In more detail, the hospitals are located near the patient house and equipped with facilities, equipments, and instruments which make it possible to conduct the biopsies, the medical imaging examinations, and the therapies. The lists of medical specialists include medical specialists relevant to the symptom of the patient, and to the diagnosis and the treatment of the patient's disease. The information may be used as useful reference information when the primary physician and the patient select a hospital, equipments, and apparatuses for the biopsies and the medical imaging examinations. The information may also be useful when the primary physician and the patient select a hospital and equipments for the therapy. Further, the information may be useful when the primary physician and the patient select the interpretation doctor and the medical specialists.

The cyber hospital agent system 102 makes the doctor/facility information request to the cyber hospital database system 104 (step 33). The cyber hospital database system 104 retrieves and makes a list of hospitals. The listed hospitals are located near the patient house and equipped with facilities, equipments, and instruments which make it possible to conduct the biopsies, the medical imaging examinations, and the therapies in accordance with the plan to which the patient has agreed. The cyber hospital database system 104 also retrieves and makes a list of medical specialists all over the world who are registered in the cyber hospital system wherever the medical specialists are located. The listed medical specialists relate to the symptom of the patient, and to the diagnosis and the therapy of the patient disease. Further, the cyber hospital database system 104 retrieves and makes a list of interpretation doctors all over the world who are registered in the cyber hospital system wherever the interpretation doctors are located. The listed interpretation doctors are appropriate for the medical imaging examinations (step 34).

The listed information is supplied with details of the information to the cyber hospital agent system 102 (step 35). Regarding each medical specialist, the details include a medical specialist's name, a personal history, a name of a hospital for which the medical specialist works, an address of the hospital, a field of specialization, the number of experienced operations, techniques, and mortality, for example. Regarding each interpretation doctor, the details include an interpretation doctor's name, a personal history, a name of a hospital for which the interpretation doctor works, an address of the hospital, a field of specialization, and the number of interpreted images, for example. Regarding each biopsy equipment, each medical imaging examination equipment, and each therapy equipment, the details include the year of manufacture, a version, an upgrade history, an installation date, equipped features, a cumulative operating time, the number of uses, a frequency in use, and a breakdown history, for example.

The cyber hospital agent system 102 prepares a GUI display window for the patient and a GUI display window for the primary physician, using the information supplied from the cyber hospital database system 104 (step 36). The GUI display window for the patient is prepared based on a part of the information, which is allowed for the patient to access. Similarly, the GUI display window for the primary physician is prepared based on a part of the information, which is allowed for the primary physician to access. The GUI display window for the patient is transmitted to the patient terminal 600 (step 37). The GUI display window for the primary physician is transmitted to the consultation room terminal 201 (step 38). The patient and the primary physician work together and study selections of the biopsy equipment, the medical imaging equipment, the therapy equipment, the medical specialist, and the interpretation doctor. As a result, one or more of biopsy equipments, medical imaging equipments, therapy equipments, medical specialist(s), and interpretation doctor(s) are determined by the patient and the primary physician (steps 39 and 40). Here, for example, a determining operation is made only by the consultation room terminal 201. Information of the determination is transmitted to the cyber hospital agent system 102 from the consultation room terminal 201 (step 41). In response to reception of the determination information, the cyber hospital agent system 102 prepares requests for acceptance and transmits the requests to the medical specialist(s) and the interpretation doctor(s) determined by the patient and the primary physicians. When the medical specialist(s) and the interpretation doctor(s) decide to accept the requests, the acceptance information is transmitted to the cyber hospital agent system 102 from consultation room terminals used by the medical specialist(s) and the interpretation doctor(s). These acceptance operations may be similar to the operations described in steps 13 to 16. Accordingly, the medical specialist(s) and the interpretation doctor(s) are reserved for the patient. The cyber hospital agent system 102 registers the determined biopsy equipment(s), medical imaging equipment(s), therapy equipments, medical specialists), and interpretation doctor(s) into the cyber hospital team as the primary physician has already been registered (step 42).

At this stage, the cyber hospital is completed for the patient. As described above, the cyber hospital may include the primary physician, the medical specialists, the interpretation doctor(s), the biopsy equipment(s), the medical imaging equipment(s), and the therapy equipment (s). The primary physician is selected from among doctors located all over the world by the determination of the patient. The medical specialists) is selected from among medical specialists located all over the world by the determination of the patient and the primary physician. The interpretation doctor(s) is selected from among interpretation doctors located all over the world by the determination of the patient and the primary physician. The biopsy equipments), the medical imaging equipment(s), and the therapy equipment(s) are selected from among equipments provided in hospitals located near the patient's house by the determination of the patient and the primary physician. Hospitals located near the patient's house include, for example, those where the patient can go by car or by a public transport without much problem.

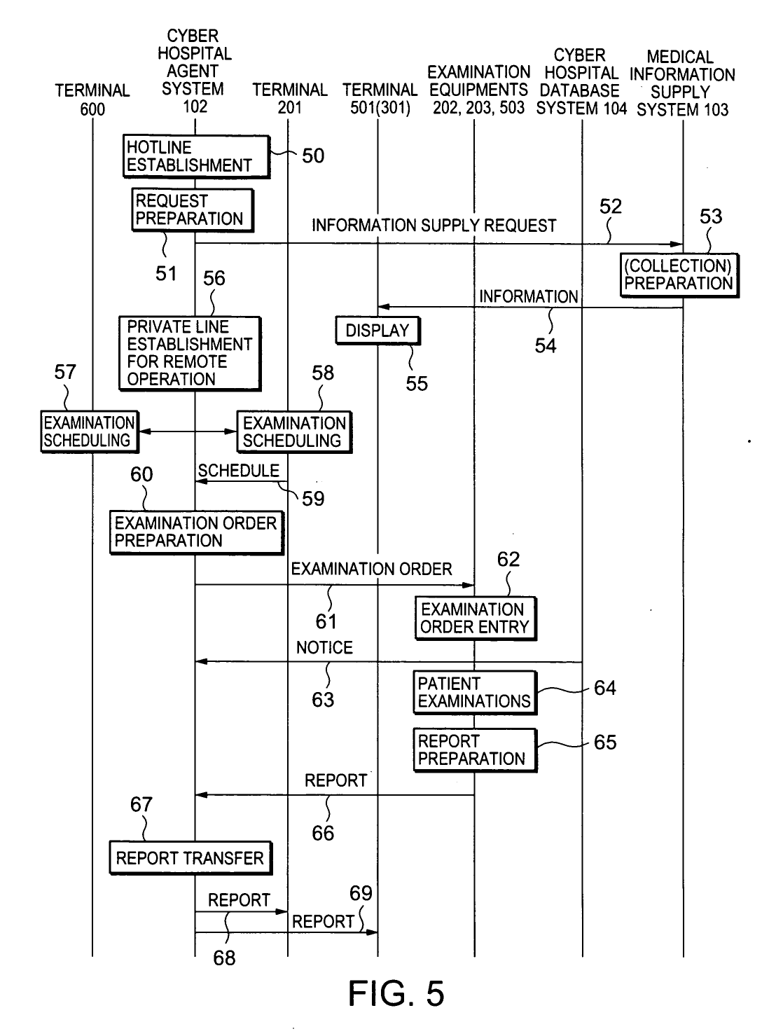

In FIG. 5, when the cyber hospital has been established, the cyber hospital agent system 102 establishes communication hotlines using, for example, IP telephones among the consultation room terminal 201 of the primary physician, the consultation room terminal 501 of the medical specialist, and the consultation room terminal 301 of the interpretation doctor when the doctor in the hospital B has been selected as the interpretation doctor. The cyber hospital agent system 102 may alternatively provides the consultation room terminals 201, 501, and 301 with information necessary for establishing similar communication hotlines (step 50).

The cyber hospital agent system 102 also prepares the information supply request (step 51) and transmits the request to the medical information supply system 103 (step 52). This is for supplying the consultation room terminals 501 and 301 with information similar to that supplied to the consultation room terminal 201 in steps 23 to 25. Accordingly, the medical information supply system 103 prepares a display window in a form of a GUI based on collected information of the patient, the deduction result, the determined actual plan, and the forecast result in accordance with the information supply request (step 53). The prepared display window including such information is transmitted to the consultation room terminals 501 and 301 in substantially or almost real time (step 54). The transmission may be implemented to the consultation room terminals 501 and 301 directly or via the cyber hospital agent system 102 from the medical information supply system 103. In the consultation room terminals 501 and 301, the GUI display window is displayed (step 55).

When the electrocardiograph 202, the magnetic resonance imaging apparatus 203, and the catheter equipment 503 are selected as the biopsy equipment, the medical imaging equipment, and the therapy equipment, the cyber hospital agent system 102 enables the consultation room terminals 201 and 501 to remotely operate the electrocardiograph 202, the magnetic resonance imaging apparatus 203, and the catheter equipment 503. For example, the cyber hospital agent system 102 establishes a private line between the consultation room terminal 201 and each of the electrocardiograph 202, the magnetic resonance imaging apparatus 203, and the catheter equipment 503. Similarly, the cyber hospital agent system 102 establishes a private line between the consultation room terminal 501 and each of the electrocardiograph 202, the magnetic resonance imaging apparatus 203, and the catheter equipment 503 (step 56).

As described before, the consultation room terminal 201 of the primary physician is connected to the patient terminal 600 of the patient through the communication hotline. Therefore, the patient and the primary physician discuss when to take the biopsy, the medical imaging examination, and the therapy, respectively. As a result, a schedule for those is determined by the patient and the primary physician (steps 57 and 58). The determined schedule is transmitted from the consultation room terminal 201 to the cyber hospital agent system 102 (step 59).

The cyber hospital agent system 102 prepares examination orders of the biopsy, the medical imaging examination, and the therapy in accordance with the transmitted schedule (step 60). The examination orders are transmitted from the cyber hospital agent system 102 to order entry systems for those examinations, respectively (step 61). In the order entry system provided for each of the electrocardiograph 202 and the magnetic resonance imaging apparatus 203 in the hospital A, each examination order is entered for reservation. Similarly, in the order entry system provided for the catheter equipment 503 in the cardiovascular center D, an examination order is entered for reservation (step 62). In response to the order entries, the order entries are informed to the cyber hospital agent system 102 (step 63). The cyber hospital agent system 102 informs the patient terminal 600 and the consultation room terminal 201 about the order entries.

Accordingly, the patient goes to hospital A on the scheduled day(s) and takes examinations by the electrocardiograph 202 and the magnetic resonance imaging apparatus 203. Similarly, the patient also goes to cardiovascular center D on the scheduled day and takes a therapy examination by the catheter equipment 503 (step 64). Based on results of the examinations, examination reports are prepared in the hospital A and the cardiovascular center D (step 65). The examination reports include a result of the electrocardiography, image data acquired by the magnetic resonance imaging apparatus 203, and a result of the catheter examination. The prepared examination reports are transmitted to the consultation room terminals 201, 501, and 301 directly or via the cyber hospital agent system 102 (steps 66 to 69) When the interpretation doctor in the hospital B receives the image data in the consultation room terminal 301, the interpretation doctor interprets the image data and prepares a medical report regarding the image data. The prepared medical report is transmitted to the consultation room terminals 201 and 501 directly or via the cyber hospital agent system 102 from the consultation room terminal 301.

Similarly, the medical specialist makes a diagnosis based on the examination reports, the medical report, the image data, the patient information, and the like. The diagnosis result is transmitted to the consultation room terminal 201 directly or via the cyber hospital agent system 102 from the consultation room terminal 501.

Any information, data, report, plan, operation, action, and determination regarding the patient is stored in appropriate databases and managed by the virtual patient system so as to be collected whenever they are needed.

As described above, the cyber hospital system according to the first embodiment of the present invention, establishes a cyber hospital particularly for a specific patient according to the patient preference or by taking the patient preference and conditions into consideration. In the cyber hospital, even if doctors, medical specialists, and interpretation doctors usually work in different places, respectively, they work together through networks as a cyber hospital team for the patient, using medical examination facilities as a part of the cyber hospital team when they are selected by the patient.

Further, the cyber hospital according to the first embodiment can be established for a patient by an ambulance crew in an ambulance even while the patient is being brought to a hospital. This may make it possible for a doctor in the hospital to obtain much of necessary information of the patient from the ambulance crew and the virtual patient system and almost know what to do with the patient in advance. That is, the doctor may be able to almost get ready for the patient prior to the patient's arrival. Particularly, since it is not rare that the patient in emergency case hardly speaks or cannot answer doctor's questions properly, the doctor can obtain much or most of necessary patient information in advance except for information to be obtained from an actual symptom of the patient.

(Second Embodiment)

The medical information supply system 103 as apart of the cyber hospital system according to the first embodiment will be described in detail below. In the following description, explanations will be made when the medical information supply system 103 includes the virtual patient system. Therefore, the second embodiment will substantially describe the virtual patient system. A term ‘medical information supply system 103’ may also be read as ‘virtual patient system’ in more than one case in the following description. The virtual patient system may also be used independently from the cyber hospital system.

In one example, the medical information supply system 103 is basically connected to a plurality of databases through networks. The networks may include an electronic network, an optical fiber network, and a radio network. Each database may be provided, for example, in a hospital, and stores information of patients. The information of the patients includes medical information, health information, and care information. The medical information supply system 103 may have a database management system which knows and manages in which database the patient information is stored. The medical information supply system 103 collects necessary information from the databases based on the database management system and requests by a user such as the patient, the doctor, the medical specialist, and the ambulance crew. The requests may include various conditions or information.

For example, the medical information supply system 103 collects necessary information based on patient identification information (hereinafter referred to as a patient ID), the user role information described before, the situation information also described before, and a symptom of the patient. Accordingly, the medical information supply system 103 can supply a user with the collected information which is useful or likely to become useful for the user.

In order to supply such useful information, the medical information supply system 103 may preferably be required the following features.

Specifying a storage place of all or as much patient information as possible regarding a health, a medical service, and a welfare from a birth to a death

Collecting information of a latest research and a guideline of a diagnosis and a treatment such as, for example, the EBM

Forecasting how the patient would become in the future by a certain medical treatment if the medical treatment was applied to the patient

Collecting information which is useful or likely to become useful for a user from a plurality of databases connected to the medical information supply system 103 through various communication lines, such as, for example, the Internet, telephone networks, optical fiber communication networks, and radio communication networks

Displaying information

Accessing to various types of networks

Recognizing the user

Specifying in what situation a user needs information and what the information is

Displaying only necessary information in a user friendly manner

Distinguishing between information allowed and not allowed to disclose to a third party

Having a security feature

Having a privacy protection feature

Displaying a virtual patient body (to be described later)

Collecting more valuable information by using various patient information as retrieval keywords than information collected by a single use of the keywords

Sorting out information corresponding to the user role of a user (e.g., a doctor, a radiological technologist, a nurse, a patient, and an insurance agent) and displaying the information in an eye friendly manner

Sorting out information based on the situation information and displaying the information in an eye friendly manner

The medical information supply system 103 is required to sort out, for example, whether a doctor uses the information when a patient is brought into a hospital in an emergency case or when a health examination is implemented.

Specifying necessary information

Determining how to present information (position, order, in two-dimension, or in three-dimension)

Recognizing whether necessary information exists or not

Specifying information which is necessary but is not stored in any database

Urging to obtain and store information which is necessary but is not stored in a database

Making it possible for a user to access to and use collected information anywhere and anytime

Informing the user of missing information and urge the user to input the missing information

collecting patient information in real time and store the collected information

The patient information includes, for example, one or more of a body temperature, a heart rate, a blood pressure, a urine examination result, a fecal examination result, an electrocardiogram, and an electrocephalogram.

When features required for the medical information supply system 103 are categorized in accordance with the user role, the features may be categorized as follows.

<When a User is a Doctor>

Making it possible for the user to display suspected part, diagnosis result information, EBM information, and a latest research result based on collected patient information

Presenting the user with a medical treatment plan which is believed to be best at that moment based on diagnosis results including a deducted disease name

Presenting the user with a most appropriate diagnosis and treatment path (a critical path) A plurality of critical paths are presented with advantages and disadvantages of each path so as to provide the user with information for his/her decision.

Numerically displaying a disease possibility when the user inputs patient's symptom information

Overlaying a plurality of medical images in a display in any manner so as to improve the merit in observation more than when each image is observed separately

Displaying pharmaceutical information

Finding a disease by comparing actual information of a patient to reference data representing standard data which could be obtained if the patient was in a healthy condition

The reference data maybe prepared by considering various conditions, such as, for example, age, sex, weight, height, smoking/non-smoking, and drinking/non-drinking. The reference data are not necessary to be standard data but may represent a condition for what a patient would like to become.

Deducing a future health condition of a patient based on a current health trend of the patient

<When a User is a Nurse or a Public Health Nurse>

Making it possible for the user to understand, communicate with, and take care of the patient as if the user knew the patient for a long time

Presenting the user withapatient's preference (e.g., a patient’ favorite baseball team)

Presenting the user with a patient's family structure, a patient's background, and a patient's hard luck story

Presenting the user with a patient's condition (e.g., glossy skin, vivid eye expression, and somehow dull looking)

Presenting the user with a patient desired nursing policy (e.g. The patient wants the user to always speak to the patient. The patient wants the user to leave the patient alone. The patient wants to go out.)

Forecasting a future health condition based on past and current health trends of a patient and making it possible for the user to instruct the patient how to prevent a disease

<When a User is a Radiological Technologist or a Doctor in Radiology>

Presenting the user with anamnesis and allergy information of a patient

Presenting the user with patient images acquired in the past, medical reports prepared in the past, and interpretation doctors' names

<When a User is a Patient>

Making it possible for the user to comprehend a current health condition

Making it possible for the user to anticipate when the user will die

Presenting the user with a current disease name

Presenting the user with a reason of the current disease

Presenting the user with a plurality of cures

Presenting the user with an expense, a period, a medical facility, a doctor's name, a risk (e.g., mortality, adverse effect), and an advantage, with respect to each of the cures

Making it possible for the user to know from which disease the user is going to suffer in the future and when it is

Presenting the user with a prevention of the disease

Collecting patient information in real time when the user suddenly upsets his/her health; automatically letting a most appropriate doctor know about the user; and instructing the user what to do if the user manages to respond

The patient information includes one or more of a height, a weight, a body temperature, a heart rate, a blood pressure, a urine examination result, a fecal examination result, an electrocardiogram, and an electrocephalogram, regarding the user. If the user cannot manage to respond to the instruction, an appropriate treatment or response is automatically implemented in accordance with seriousness of the upset and an ideal treatment which the user believes.

Making it possible for the user to recognize a user's ideal body and presenting the user with how to make that body

Caring a mental health of the user

Introducing a person who cares a mental health to the user

Listening to and advising the user

Buying and selling a right to have a check-up as a patient in a particular hospital

Disclosing a user's disease condition to a plurality of hospitals and putting a care of the user out to tender

The care may include, for example, an expense, a period, a quality of an admission facility, and amenity.

<When a User is an Insurance Agent>

Forecasting a future health condition based on past and current health trends of a patient and calculating a premium

Presenting the user with an expense, a period, a hospital facility, a doctor's name, and a risk, regarding each cure

Making it possible for the user to buy and sell a right to have a check-up as a patient in a hospital

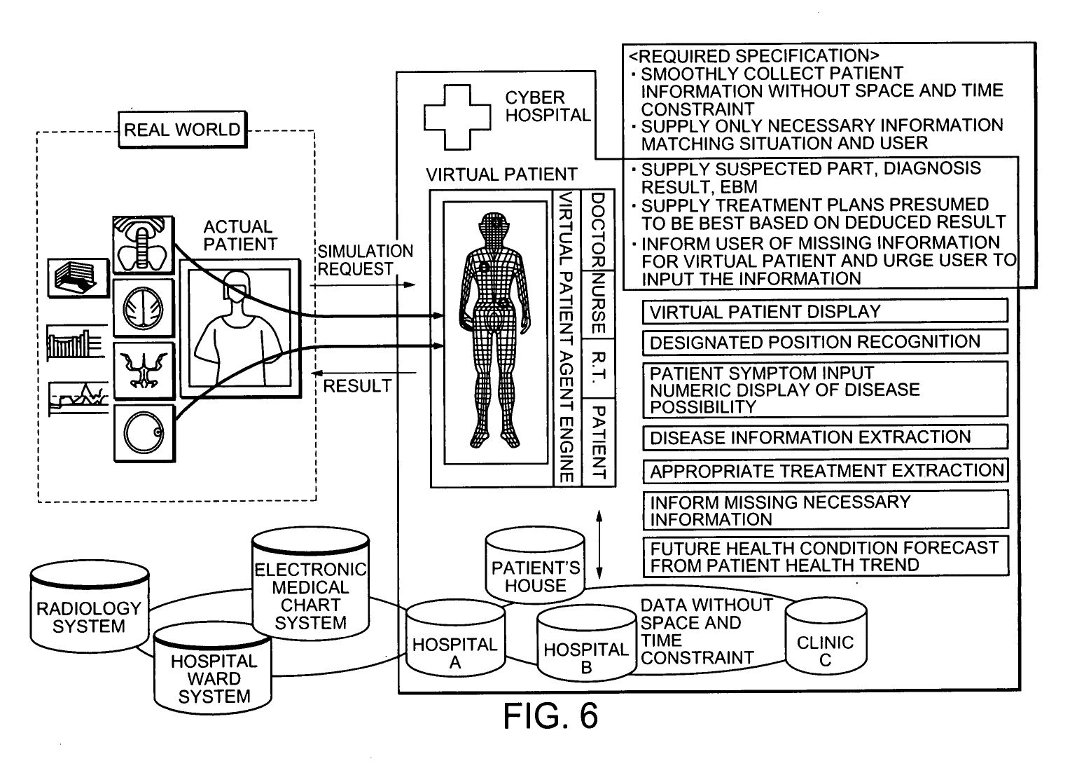

FIG. 6 is an illustration showing a first exemplary schema of the medical information supply system 103 according to the second embodiment of the present invention. When the medical information supply system 103 operates the virtual patient system as a part of its features, a virtual patient agent engine included in the virtual patient system is rendered operative so as to control the virtual patient system. Similarly, the virtual patient agent engine is also rendered operative when the virtual patient system is used independently from or in connection with the medical information supply system 103.

The virtual patient agent engine may be required the following specifications, for example.

It is possible to smoothly collect all or most of the medical information of a patient without a space and time constraint.

It is possible to supply only necessary information which matches a use situation (scene) and a user of the information.

It is possible to supply suspected part, diagnosis result information, and EBM information based on the patient information.

It is possible to supply a plurality of treatment plans which are presumed to be best at that moment based on deducted diagnosis results.

It is possible to inform a user of missing information for a virtual patient and urge the user to input the missing information.

In addition to the above specifications, the virtual patient agent engine may also be required the following features.

Displaying a virtual patient

Recognizing a designated position in the displayed virtual patient

Inputting a patient symptom and numerically display a disease possibility

Extracting disease information of a specific

Extracting most appropriate treatment

Informing a user of missing information which is necessary for a diagnosis

Forecasting a future health condition based on past and current health trends of a patient

As shown in FIG. 6, patient information which has actually been obtained by examinations is stored in various databases and memory devices provided in various systems, such as, for example, a radiology system, an electronic medical chart system, and a hospital ward system in each of the hospitals A and B, and maybe the clinic C, and maybe in the patient's house in a decentralized manner. The virtual patient agent engine manages the patient information stored in those databases. The stored information is used without a space and time constraint. When the patient information of a specific patient is collected by the virtual patient agent engine, the virtual patient of the specific patient is prepared as a gathering of the patient information. A virtual patient body to be displayed in a user's terminal display may correspond to the virtual patient.

The virtual patient and accompanying patient information is accessed differently, depending on a user role (e.g., a doctor, a nurse, a radiological technologist, and a patient) since accessible information is restricted according to the user role.

A doctor may request a simulation based on the patient information to the medical information supply system 103. Alternatively, the medical information supply system 103 may automatically implement the simulation such as the deduction, the preparation, and the forecast in response to the collection of the patient information as described before. Since the patient information including the virtual patient and the simulation result is transmitted to the doctor, the doctor studies the patient information and implements or plans an actual examination.

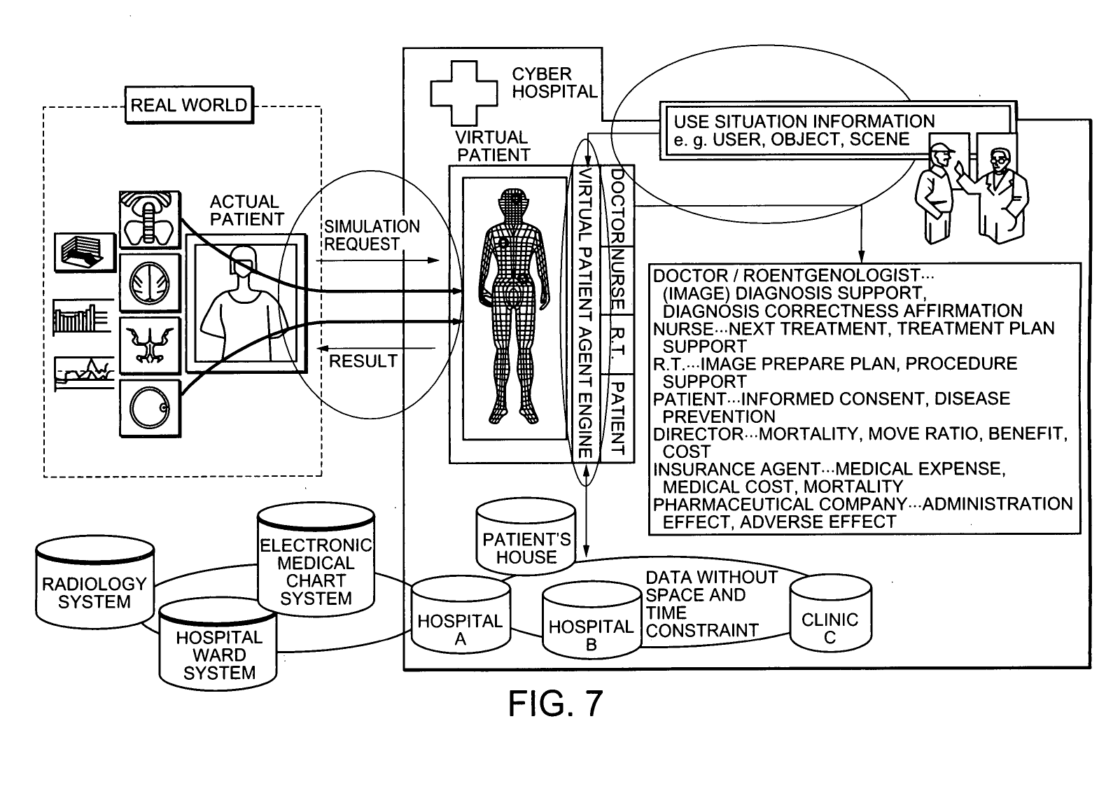

FIG. 7 is an illustration showing a second exemplary schema of the medical information supply system 103 according to the second embodiment of the present invention.

The virtual patient agent engine has a feature of collecting necessary information from a great amount of information stored in the databases according to a user role and a feature of supplying the collected information to the user as basic features.

For example, when a user is a doctor or a roentgenologist, the virtual patient agent engine supplies, for example, (i) information which can support a diagnosis, (ii) information which the user can affirm a correctness of a diagnosis result, and (iii) information which can support an image diagnosis. When a user is a nurse, the virtual patient agent engine supplies, for example, (i) information of a next nursing treatment and (ii) information which can support a treatment plan. When a user is a radiological technologist, the virtual patient agent engine supplies, for example, (i) information of an image preparation plan and (ii) information which can support procedures. When a user is a patient, the virtual patient agent engine supplies, for example, (i) information of an informed consent and (ii) information of knowledge for preventing a disease. When a user is a hospital director, the virtual patient agent engine supplies, for example, information of mortality, a hospital move ratio, a benefit, and a cost which are helpful for a hospital management. When a user is an insurance agent, the virtual patient agent engine supplies, for example, information of a medical expense, a medical cost, and mortality. When a user is a pharmaceutical company, the virtual patient agent engine supplies, for example, information of an administration effect and an adverse effect of medicine.

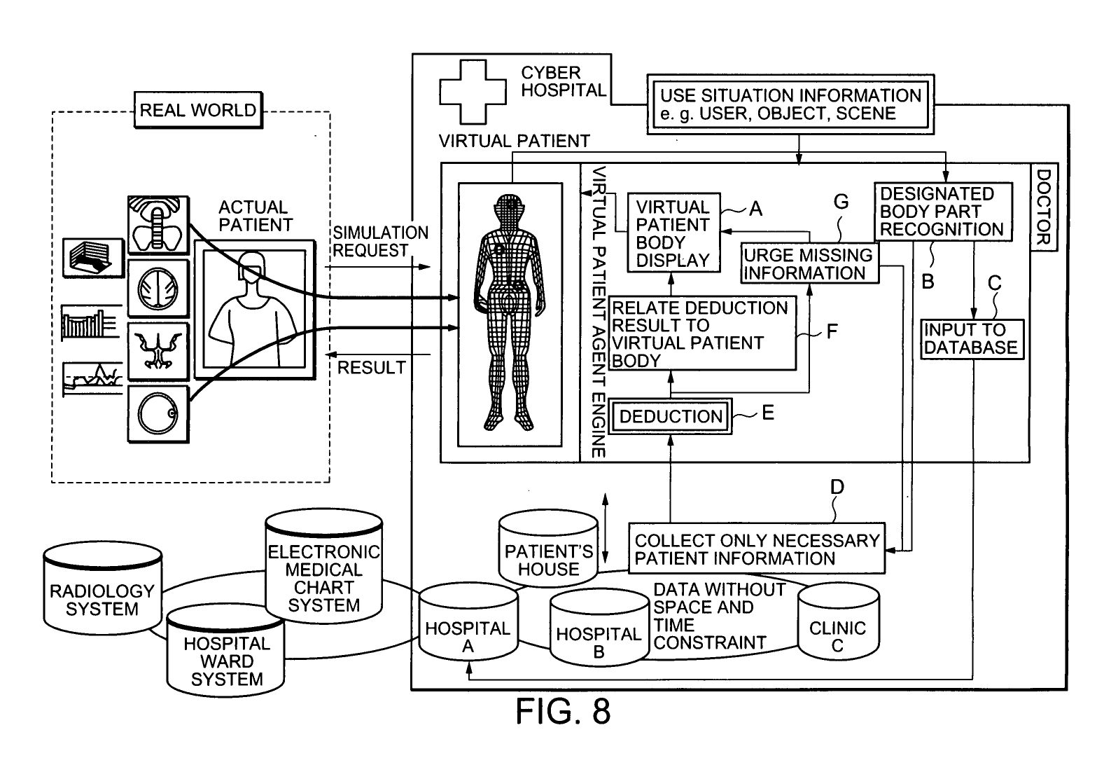

FIG. 8 is an illustration showing a third exemplary schema of the medical information supply system 103 according to the second embodiment of the present invention. FIG. 8 shows a brief relationship among some features of the medical information supply system 103.

This is an example when a user is a doctor. A plurality of virtual patient bodies are prepared in advance in accordance with a sex, an age, a body build and the like. The virtual patient body to be displayed is selected based on information input by the doctor. The selected virtual patient body is accordingly displayedinadoctor's consultation room terminal (step A). When the doctor moves a cursor onto a certain part of the virtual patient body and clicks on the part for designation, the medical information supply system 103 recognizes the designated part (step B). The designated part information is input to the database (step C). The medical information supply system 103 then collects (or extracts) necessary information of the patient relating to the designated part from the databases and memory devices connected to the medical information supply system 103 (step D). Based on the collected patient information, the medical information supply system 103 deduces a disease name of the patient (step E). The deduced disease name is related or linked to the designated part of the virtual patient body. Further, the designated part is marked on the virtual patient body (step F). In the above deduction, however, when the medical information supply system 103 determines that there is some missing information for implementing the deduction, a message is displayed in the doctor's consultation room terminal so as to urge the doctor to input the missing information (step G) In response to the doctor's input of the missing information, the medical information supply system 103 deduces a disease name of the patient. When the above operation is repeated, a plurality of designated parts may be marked on the virtual patient body. According to the medical information supply system 103, the doctor can efficiently obtain various information of the patient through the virtual patient body and know the deducted disease name as a suspected disease of the patient.

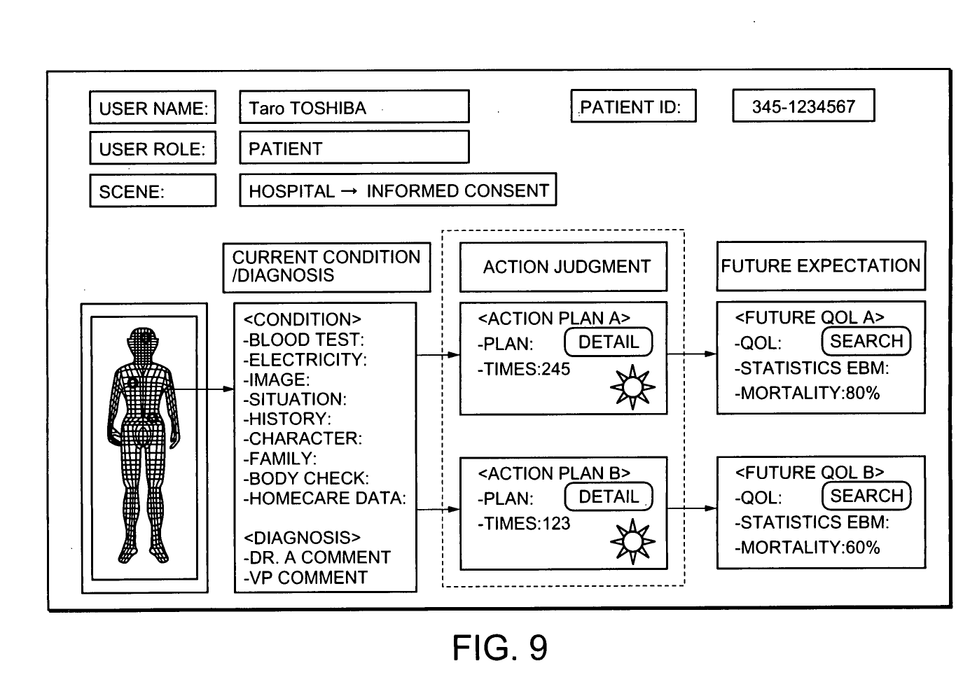

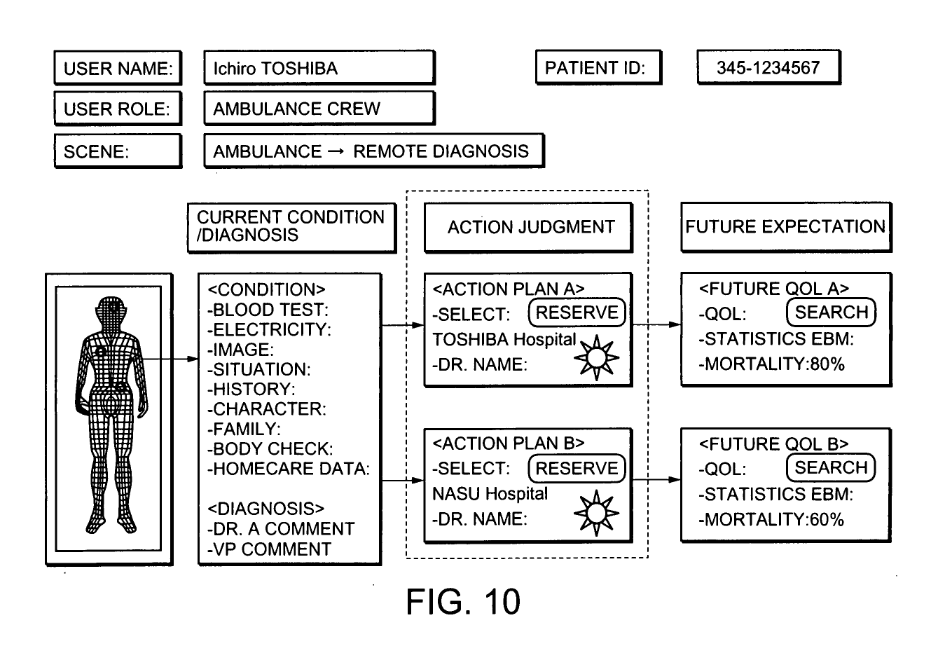

FIGS. 9 and 10 are illustrations showing examples of display window displayed in a user's terminal according to the second embodiment of the present invention. FIG. 9 shows a case that a user is a patient. FIG. 10 shows a case that a user is an ambulance crew. When a user is going to use the medical information supply system 103, the user is requested to input a user name, a patient ID, user role information, and situation (scene) information.

As shown in FIG. 9, a user name ‘Taro TOSHIBA’ is input as the user name. Since this user is a patient, this user's patient ID is input as the patient ID and ‘patient’ as the user role information. The situation is supposed to be a case that the patient has a look at information when a doctor explains some treatment plans to the patient for an informed consent.

When one (e.g., chest) of marked parts on the virtual patient body is clicked on, a list of patient information regarding the chest of ‘Taro TOSHIBA’ is displayed in a display field ‘Current Condition/Diagnosis’. When an item is clicked on, detailed information of the item is displayed in a separate window (not shown in FIG. 9). Next to the display field ‘Current Condition/Diagnosis’, another information is displayed as ‘Action Judgment’. In a display field ‘Action Judgment’, a plurality of action plans for the patient treatment are displayed with ‘Detail’ buttons. By clicking on the ‘Detail’ button in each plan, the treatment plan is displayed in detail in a separate window (not shown in FIG. 9). Further, ‘Future Expectation’ information is displayed for each plan. The ‘Future Expectation’ shows a future condition of how the patient would become if the presented action plan was implemented on the patient. For example, a quality of life (QOL), EBM, and mortality are displayed in the display field. Similar to the ‘Action Judgment’, when a button ‘Search’ is clicked on, detailed information is displayed in a separate window.

In FIG. 10, a user name ‘Ichiro TOSHIBA’ is input as the user name. When the ambulance crew would like to obtain information of the patient ‘Taro TOSHIBA’, his patient ID is input by the ambulance crew. Since this user is an ambulance crew, ‘ambulance crew’ is input as the user role information. The situation is supposed to be a case that the ambulance crew obtains thepatient's information in an ambulance so as to implement a first aid or to know which hospital is most suitable to bring the patient in, for example.

Similar to FIG. 9, when one (e.g., chest) of marked parts on the virtual patient body is clicked on, a display field ‘Current Condition/Diagnosis’ is displayed along with a display field ‘Action Judgment’. In the display field ‘Action Judgment’, a plurality of action plans for bringing the patient into a hospital are displayed with ‘Reserve’ buttons. Each plan shows a destination hospital name and a name of a doctor in charge. By clicking on the ‘Reserve’ button in each plan, a reservation is made for the destination hospital. Further, similar to FIG. 9, ‘Future Expectation’ information is displayed for each plan.

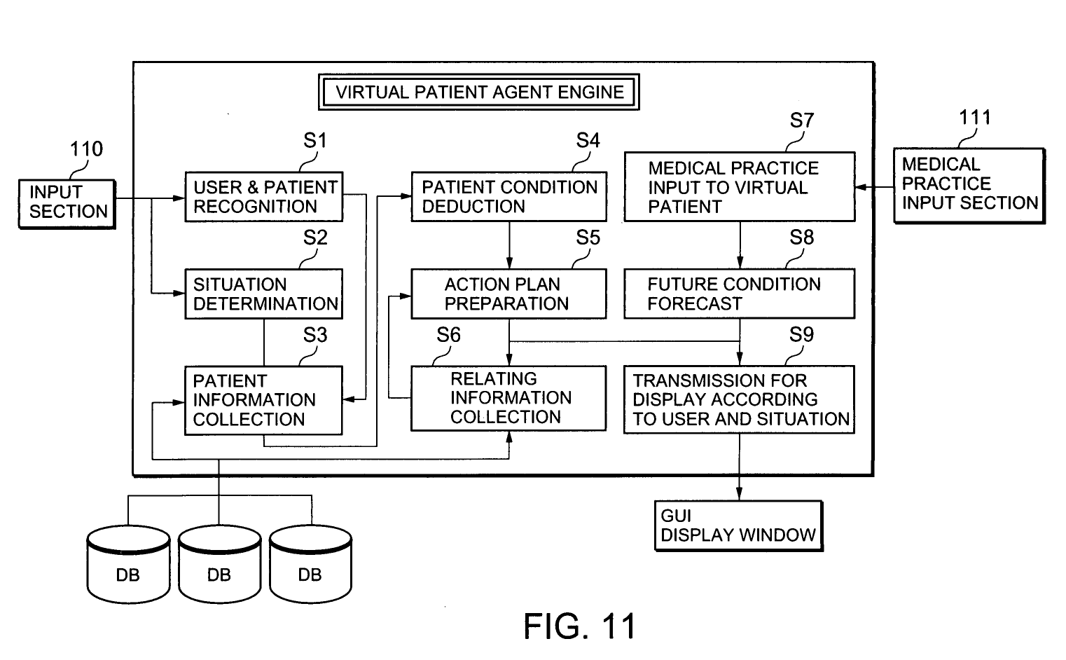

FIG. 11 is an illustration showing an example of a processing flow in the virtual patient agent engine according to the second embodiment of the present invention.

When a user inputs a user name, a patient ID, user role information, and situation information from a user's terminal as shown in FIG. 9 or 10, such input information is transmitted through networks and is input to an input section 110 provided in the medical information supply system 103. The input information is taken into or input to the virtual patient agent engine. Accordingly, the user and the patient are recognized based on the user name, the user role information, and the patient ID (step S1). Also a situation of an information use is determined based on the user role information and the situation information (step S2). The virtual patient agent engine then collects information of the patient which is appropriate for the user role in the determined situation (step S3). The information collection is implemented against a plurality of databases provided in hospitals through networks.

The virtual patient agent engine deduces a disease name which is suspected for the patient as a patient condition based on the collected patient information (step S4). Appropriate action plans are prepared based on the deduced disease name or condition (step S5). For the preparation of the action plans, another information which is necessary for the action plans is collected from the same or different plurality of databases (step S6). Such another information may relate to diagnoses and treatments to be included in the action plans. The collected another information is fed to step S5.

Meanwhile, a doctor (or a primary physician) may input medical practice information from a doctor's terminal. The medical practice information may be what the doctor is actually planning to implement as examinations and/or treatments. Such input information is transmitted through networks and is input to a medical practice input section 111 provided in the medical information supply system 103. The input information is taken into or input to the virtual patient agent engine and is input as apart of the virtual patient (step S7). Accordingly, the virtual patient agent engine forecasts a future condition of the patient if the doctor actually implemented a medical practice represented in the medical practice information, respectively (step S8).

The disease name deduced in step S4, the action plans prepared in step S5, and the future condition forecasted in step S8 are sorted out according to the user role information and the situation information. The sorted out information is transmitted to the user's terminal for a display (step S9) and is displayed in the user's terminal in a GUI form.

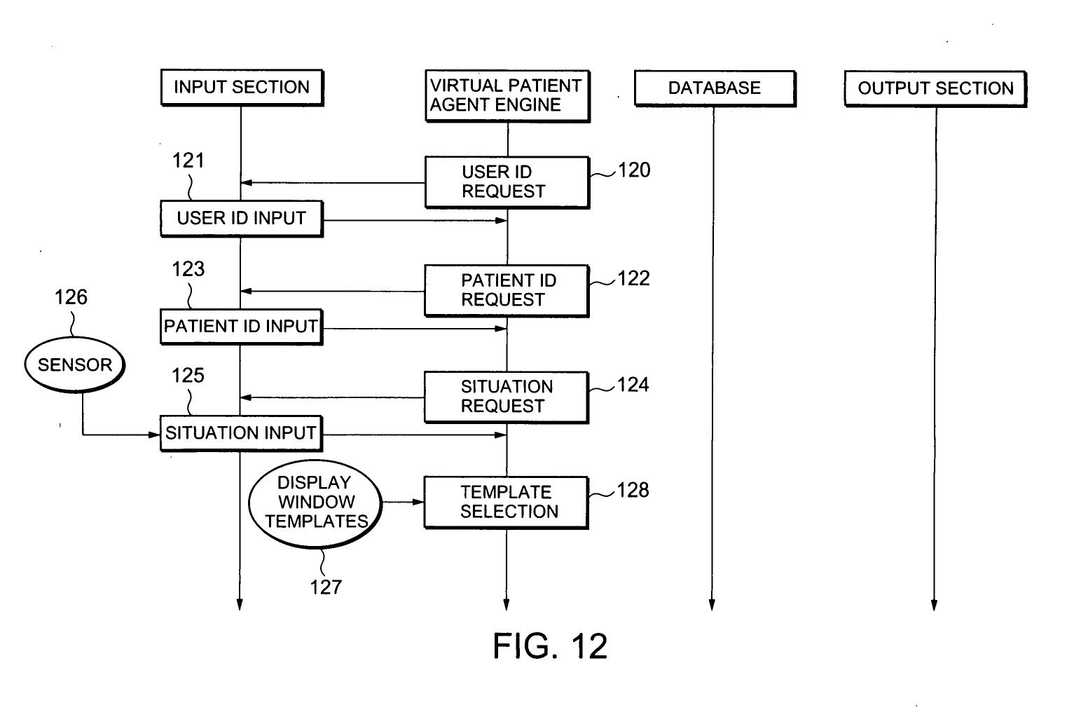

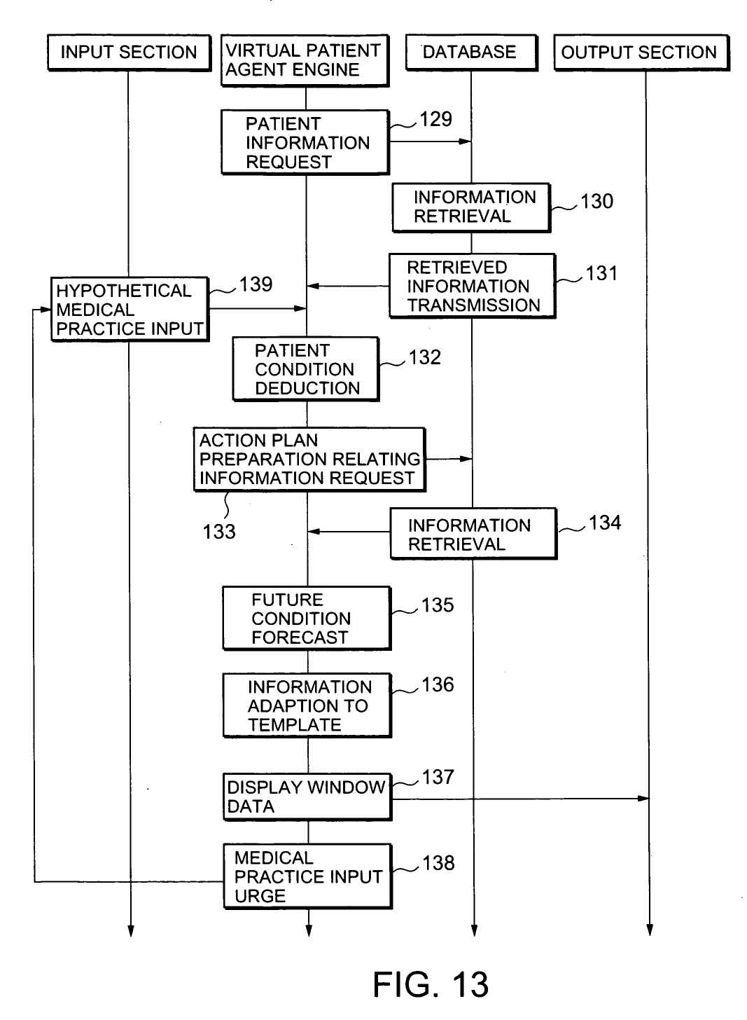

FIGS. 12 and 13 are flowcharts showing an example of a detailed processing flow regarding the virtual patient agent engine according to the second embodiment of the present invention. The flowchart shows a relationship among an input section, the virtual patient agent engine, databases, and an output section. The input section and the output section are included in the medical information supply system 103.

In FIG. 12, to use the medical information supply system 103, a user is requested to input a user ID for identifying the user by the virtual patient agent engine (step 120). In response to the request, the user inputs a user ID from a user's terminal. The input user ID is transmitted into the input section through networks (step 121). The user may be requested to input a user name, instead of the user ID. The virtual patient agent engine may also request the user to input user role information.

When the virtual patient agent engine authenticates the user ID as a permitted user ID, the virtual patient agent engine recognizes the user and the user role based on the transmitted information. The virtual patient agent engine then requests the user to input a patient ID of a patient of whom the user desires information (step 122). In response to the request, the user inputs a patient ID from the users terminal. The input patient ID is transmitted into the input section through the networks (step 123). The transmitted patient ID is received by the virtual patient agent engine. The virtual patient agent engine may alternatively request the user to input patient information including a current patient condition. The virtual patient agent engine also requests the user to input situation information (step 124). In response to the request, the user inputs situation information from the user's terminal. The input situation information is transmitted into the input section through the networks (step 125). The situation information may be input by a user's manual operation. Alternative way of the input may be made by a sensor provided in the user's terminal or connected to the user's terminal around the user (step 126). In this case, the situation information may be automatically transmitted by the sensor so that the user does not have to operate the user's terminal. The situation information transmitted by the sensor represents a place where the user is located.

When the virtual patient agent engine receives the situation information, an appropriate template of a display window is selected from a plurality of templates (step 127) prepared in advance in accordance with the user information, the user role information, and the situation information input from the user (step 128).

Further in FIG. 13, the virtual patient agent engine specifies types of information appropriate for the user role and the situation. The virtual patient agent engine transmits a request to a plurality of databases for collecting information of the patient regarding the specified information types (step 129). Each of the plurality of databases receives the request and retrieves requested information from information stored in the database based on the patient ID and the specified information types (step 130). As a result of the retrieval, each database transmits the retrieved information to the virtual patient agent engine (step 131).

The virtual patient agent engine receives (or collects) the retrieved information and deduces a patient condition such as a disease name based on the received information and other information, for example, which is directly obtained from the patient such as a symptom of the patient (step 132). This deduction may be executed by a nuero computer. The virtual patient agent engine then prepares a plurality of action plans (or a plurality of candidate action plans) with respect to a diagnosis and a treatment. The virtual patient agent engine transmits a request to a plurality of databases for collecting information relating to the prepared action plans (step 133). Each database retrieves requested information from information stored in the database (step 134). The retrieved information is transmitted to and collected by the virtual patient agent engine.

In the virtual patient agent engine, the candidate action plans may be refined to most appropriate action plans based on the collected information. Further, a future condition of the patient corresponding to each prepared action plan is forecasted with reference to the collected information relating to the candidate action plans. In other words, the virtual patient agent engine forecasts how the patient would become in the future if each of the (most appropriate) candidate action plans was implemented on the patient (step 135). The plurality of (most appropriate) candidate action plans are adapted to the selected display window template (step 136). The future conditions may be adapted as future conditions. For displaying the information-adapted template display window in a GUI form (GUI display window), data of the GUI display window are sent to the output section (step 137). The GUI display window is transmitted from the output section to the user's terminal and displayed in a manner like FIG. 9 or 10.

When the user clicks on the ‘Detail’ button in the GUI display window, such click-on information is input to the virtual patient agent engine. The virtual patient agent engine sends the collected information relating to the clicked action plan to the output section. Accordingly, the collected information relating to the clicked action plan is transmitted to the user's terminal and displayed as the detail information.

Meanwhile, the virtual patient agent engine can also display a message or an input display window so as to urge or allow the user to input medical practice information (step 138). The medical practice may be what the user is thinking of actually implementing on the patient. Therefore, the medical practice may alternatively be expressed as a medical plan or a hypothetical medical practice. When the user inputs such hypothetical medical practice information (step 139), the virtual patient agent engine implements steps 132 to 137 with respect to the hypothetical medical practice. Accordingly, the GUI display window is displayed in the user's terminal. The GUI display window includes a plurality of concrete action plans for implementing the hypothetical medical practice. Future expectation for each action plan is also included in the GUI display window.

In the second embodiment, when the medical information supply system is accessed by the cyber hospital agent system 102, such access becomes an input by the user described above. Therefore, the collected information or the GUI display window is transferred to the patient terminal 600, the consultation room terminals 201, 301, and/or 501 from the cyber hospital system.

As described above, the virtual patient agent engine as a part of the medical information supply system 103 specifies (or narrows down) types of information which may be necessary for the user according to the user role and the situation. The virtual patient agent engine then requests a retrieval of such specific information to a plurality of databases and collects the retrieved information. The virtual patient agent engine also deduces a disease name, prepares a plurality of action plans, and forecasts future expectations corresponding to the action plans.

(Third Embodiment)

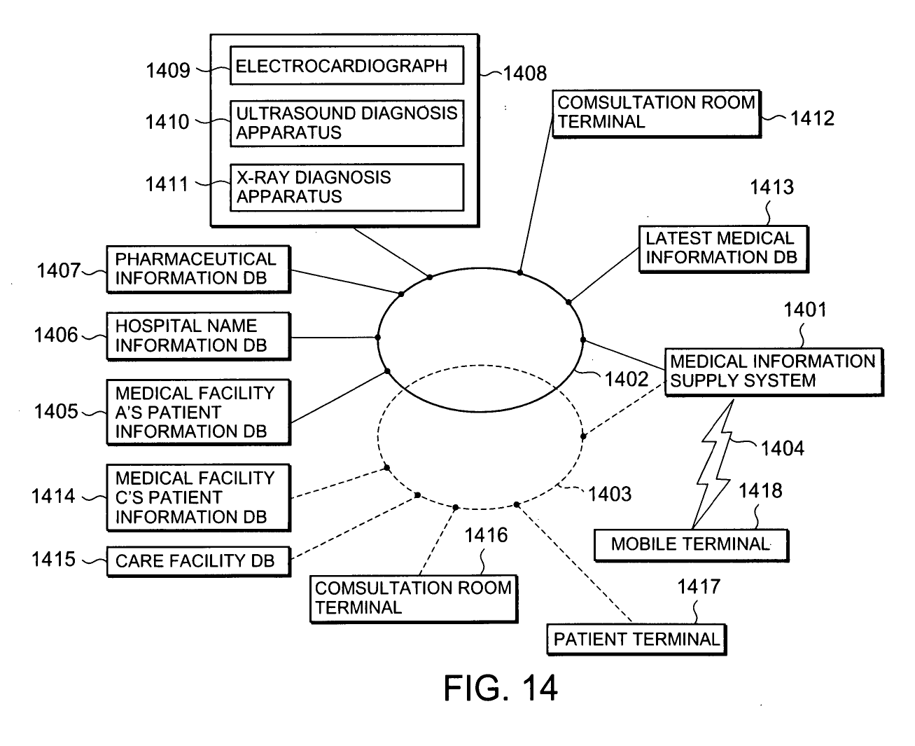

FIG. 14 shows a case that the medical information supply system is used independently. Therefore, the cyber hospital system is not necessarily required in the third embodiment although the medical information supply system may be used under a circumstance that the cyber hospital system exists. According to the third embodiment, the medical information supply system may be similar to the virtual agent system.

As shown in FIG. 14, a medical information supply system 1401 is connected to a plurality of databases and user's terminals through networks 1402, 1403, and a radio network 1404. The networks 1402 and 1403 may be electronic networks, optical fiber networks, or any other possible networks. For example, the medical information supply system 1401 is connected through the network 1402 to a pharmaceutical information database 1407, a hospital name information database 1406, a medical facility A's patient information database 1405, an information system 1408 in a hospital B, a consultation room terminal 1412, and a latest medical information database 1413 in a research institute. The medical information supply system 1401 is also connected through the network 1403 to a medical facility C's patient information database 1414, a care facility database 1415, a consultation room terminal 1416, and a patient terminal 1417 in a patient's house. The medical information supply system 1401 is still further connected to a mobile terminal 1418, for example, in an ambulance through the radio network 1404. The information system 1408 in a hospital B includes an electrocardiograph 1409, an ultrasound diagnosis apparatus 1410, and an X-ray diagnosis apparatus 1411. The medical information supply system 1401 is directly or indirectly connected to the electrocardiograph 1409, the ultrasound diagnosis apparatus 1410, and the X-ray diagnosis apparatus 1411 so as to collect medical data and images showing a patient's current condition. The consultation room terminals 1412 and 1416 may be provided in consultation rooms in hospitals and used by doctors. According to the above network connections, the medical information supply system 1401 can collect necessary information from those connected to the medical information supply system 1401.

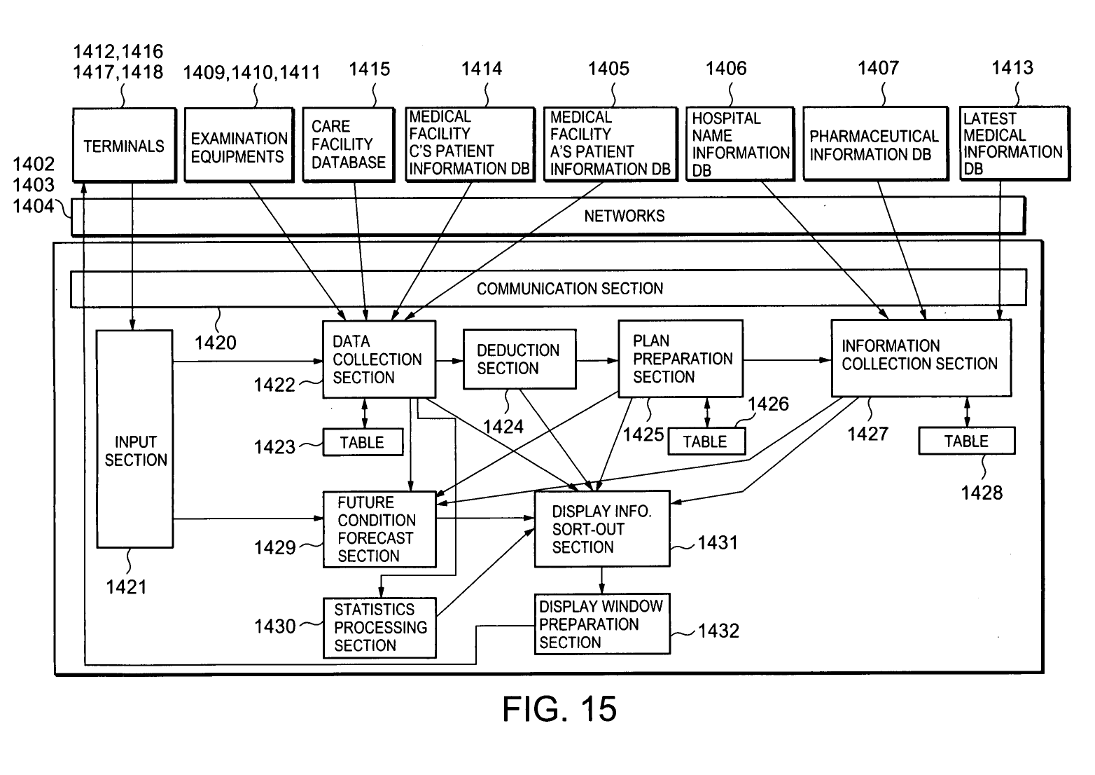

FIG. 15 is a block diagram showing an exemplary configuration of the medical information supply system 1401 according to the third embodiment of the present invention. The disclosed configuration may also be applied to a configuration of the medical information supply system 103 in the first and second embodiments.

As shown in FIG. 15, the medical information supply system 1401 includes a communication section 1420, an input section 1421, a data collection section 1422, a table 1423, a deduction section 1424, a plan preparation section 1425, a table 1426, an information collection section 1427, a table 1428, a future condition forecast section 1429, a statistics processing section 1430, a display information sort-out section 1431, and a display window preparation section 1432.

The communication section 1420 includes a plurality of communication devices corresponding to the networks 1402 to 1404. The input section 1421 takes in or inputs various types of information from the consultation room terminals 1412, 1416, the patient terminal 1417, and the mobile terminal 1418 through the networks 1402 to 1404 and the communication section 1420. The various types of information includes, for example, initial information, action plan selection information, and information regarding medical practice such as a diagnosis or a treatment. The initial information includes, for example, patient identification information such as a patient name or a patient ID, a patient's symptom, user role information, situation information, and an authentication code such as a user ID. The input section 1421 also sorts out information for urging a user to input necessary information. The input section 1421 further authenticates the authentication code.

The data collection section 1422 specifies types of information which the user may need in accordance with the user role information, the situation information, and the patient symptom. The data collection section 1422 collects information of the patient regarding the specified information types from the electrocardiograph 1409, the ultrasound diagnosis apparatus 1410, and the X-ray diagnosis apparatus 1411, the medical facility A's patient information database 1405, the medical facility C's patient information database 1414, and the care facility database 1415. The data collection section 1422 is connected to the table 1423. The table 1423 is, for example, made of a read only memory (ROM) or a non-volatile memory. To support the specifying operation by the data collection section 1422, the table 1423 may provide the data collection section 1422 with the types of information in response to inputs of the patient's symptom, the user role information, and the situation information.

The deduction section 1424 deduces a patient condition such as a name of disease from which the patient may be suffering in accordance with the information collected by the data collection section 1422. In one example, the deduction section 1424 may include a neuro computer as a deduction engine. The neuro computer may take in or input information of patient's symptom, sex, age, body build, lifestyle, anamnesis, current hospital record, blood examination result, and the like. As a result, the neuro computer outputs the disease name.

The plan preparation section 1425 prepares one or more candidate action plans, based on the deduced disease name, with respect to, for example, a diagnosis, a treatment, a first aid in an emergency case, and a cure which the user should implement next. The one or more candidate action plans may almost be determined in accordance with the user role information and the situation information. The plan preparation section 1425 is connected to the table 1426. The table 1426 is, for example, made of a read only memory (ROM) or a non-volatile memory. To support the preparation by the plan preparation section 1425, the table 1426 may provide the plan preparation section 1425 with candidate action plans in response to inputs of the deduced disease name, the user role information, and the situation information.

The information collection section 1427 collects information relating to the prepared candidate action plans from the hospital name information database 1406, pharmaceutical information database 1407, and the latest medical information database 1413. Each of the prepared candidate action plans includes information of at least one treatment. The information collection section 1427 is connected to the table 1428. The table 1428 is, for example, made of a read only memory (ROM) or a non-volatile memory. The table 1428 may provide the information collection section 1427 with at least one type of information in response to inputs of the treatment information included in the candidate action plan.

The future condition forecast section 1429 forecasts a future condition of the patient (or a future expectation) under each candidate action plan. In other words, the future condition forecast section 1429 forecasts how the patient would become in the future if at least one of treatments included in each candidate action plan was implemented on the patient. The future condition forecast section 1429 may include a neuro computer as a forecast engine. The neuro computer may take in or input information of patient's symptom, sex, age, body build, life style, anamnesis, current hospital record, blood examination result, treatment, a type of medicine to be administered, a dose of the medicine, and a latest medical information. As a result, the neuro computer outputs a QOL, an EBM, and mortality as a patient future expectation.

The statistics processing section 1430 processes information collected by the data collection section 1422. The statistically processed information is supplied to the display information sort-out section 1431.

The display information sort-out section 1431 sorts out the information collected by the data collection section 1422, the disease name deduced by the deduction section 1424, the candidate action plans prepared by the plan preparation section 1425, the relating information collected by the information collection section 1427, the future conditions forecasted by the future condition forecast section 1429, and the information statistically processed by the statistics processing section 1430, in accordance with the user role information, the situation information, and an access authority given to the user.

The display window preparation section 1432 adapts the sorted out information to a display window template so as to prepare a display window. The display window may be prepared in a GUI form. Data of the prepared display window are transmitted from the communication section 1420 to the consultation room terminals 1412, 1416, the patient terminal 1417, or the mobile terminal 1418 through one of the networks 1402 to 1404. The transmitted data are displayed in the received terminal.

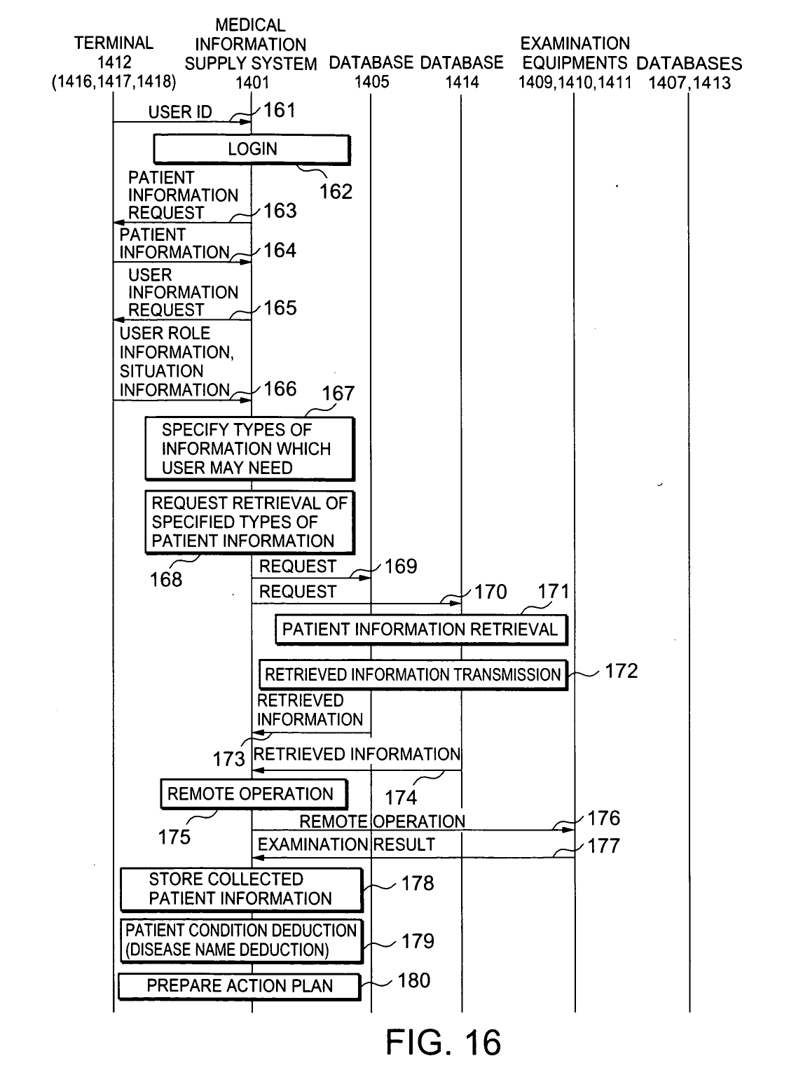

The medical information supply system 1401 configured as described above operates as follows. FIGS. 16 to 18 are flowcharts showing an exemplary operation flow of the medical information supply system 1401 according to the third embodiment of the present invention. The flowcharts shown in FIGS. 16 to 18 may be similar to those shown in FIGS. 12 and 13. The flowcharts shown in FIGS. 16 to 18, however, show further detailed operation of the medical information supply system and, accordingly, can be understood as another flowchart example.

The flowchart shows an operation relationship among, for example, the medical information supply system 1401, the consultation room terminal 1412 (, the consultation room terminal 1416, the patient terminal 1417, or the mobile terminal 1418), the medical facility A's patient information database 1405, the medical facility C's patient information database 1414, one or more of the electrocardiograph 1409, the ultrasound diagnosis apparatus 1410, and the X-ray diagnosis apparatus 1411, the latest medical information database 1413, and the pharmaceutical information database 1407.

As shown in FIG. 16, a user (for example, a doctor with the consultation room terminal 1412) inputs a user ID from the consultation room terminal 1412 so as to request a login to the medical information supply system 1401. The input user ID is transmitted to the input section 1421 of the medical information supply system 1401 (step 161). The user may be requested to input a user name, instead of the user ID. The input section 1421 authenticates the user ID and allows the consultation room terminal 1412 to login the medical information supply system 1401 (step 162). After the login, the input section 1421 requests the consultation room terminal 1412 to input patient information (step 163). In response to the request, the user inputs information of a patient ID, patient's name, sex, age, and symptom in the consultation room terminal 1412. The input patient information is transmitted to the input section 1421 (step 164). Upon the reception of the patient information, the input section 1421 then requests the consultation room terminal 1412 to input user role information and situation information (step 165). In response to the request, the user inputs user role information of the user and situation information regarding a user's information use situation (or place). The input information is transmitted from the consultation room terminal 1412 to the input section 1421 (step 166).

When the input section 1421 receives the information from the consultation room terminal 1412, the input section 1421 outputs a data collection trigger to the data collection section 1422. The data collection section 1421 specifies types of information which the user may need in accordance with the user role information, the situation information, and the patient symptom (step 167). The data acquisition section 1422 requests all or part of the medical facilityA's patient information database 1405, the medical facility C's patient information database 1414, and the like to retrieve and supply information of the patient regarding the specified information types (step 168). For example, the supply request is transmitted to the medical facility A's patient information database 1405 (step 169). Similarly, the supply request is transmitted to the medical facility C's patient information database 1414 (step 170). The medical facility A's patient information database 1405 and the medical facility C's patient information database 1414 retrieves necessary information from stored information in accordance with the request (step 171). As a result of the retrieval, the medical facility A's patient information database 1405 and the medical facility C's patient information database 1414 transmit the retrieved information to the data collection section 1422 (steps 172 to 174).

In addition to the above request, when the data collection section 1422 recognizes types of information regarding current examination information of the patient, the data collection section 1422 makes a request to supply a result of the examination in a manner similar to the above. The request may be made, for example, to the electrocardiograph 1409, the ultrasound diagnosis apparatus 1410, and/or the X-ray diagnosis apparatus 1411 provided in a hospital where the patient is currently located or in an ambulance if the patient is being brought by the ambulance. If the examination has not been implemented yet, the data collection section 1422 may request to implement the examination. Further, a remote operation request may be made to the electrocardiograph 1409, the ultrasound diagnosis apparatus 1410, and/or the X-ray diagnosis apparatus 1411. When the remote operation is implemented (steps 175 and 176), a result of the examination is transmitted from the electrocardiograph 1409, the ultrasound diagnosis apparatus 1410, and/or the X-ray diagnosis apparatus 1411 (step 177). The transmitted result is colleted by the data collection section 1422, and added as a part of the collected patient information.

The collected patient information is stored in the data collection section 1422 (step 178). The deduction section 1423 deduces a patient condition such as a disease name based on the patient information input by the user and the collected patient information including the examination result (step 179). The collected information may include, for example, patient's anamnesis, chronic illness, life style, and blood information. The plan preparation section 1425 prepares one or more candidate action plans which the user should implement next (step 180). The preparation is made based on the deduced disease name, with respect to, for example, a diagnosis, a treatment, a first aid in an emergency case, and a cure.

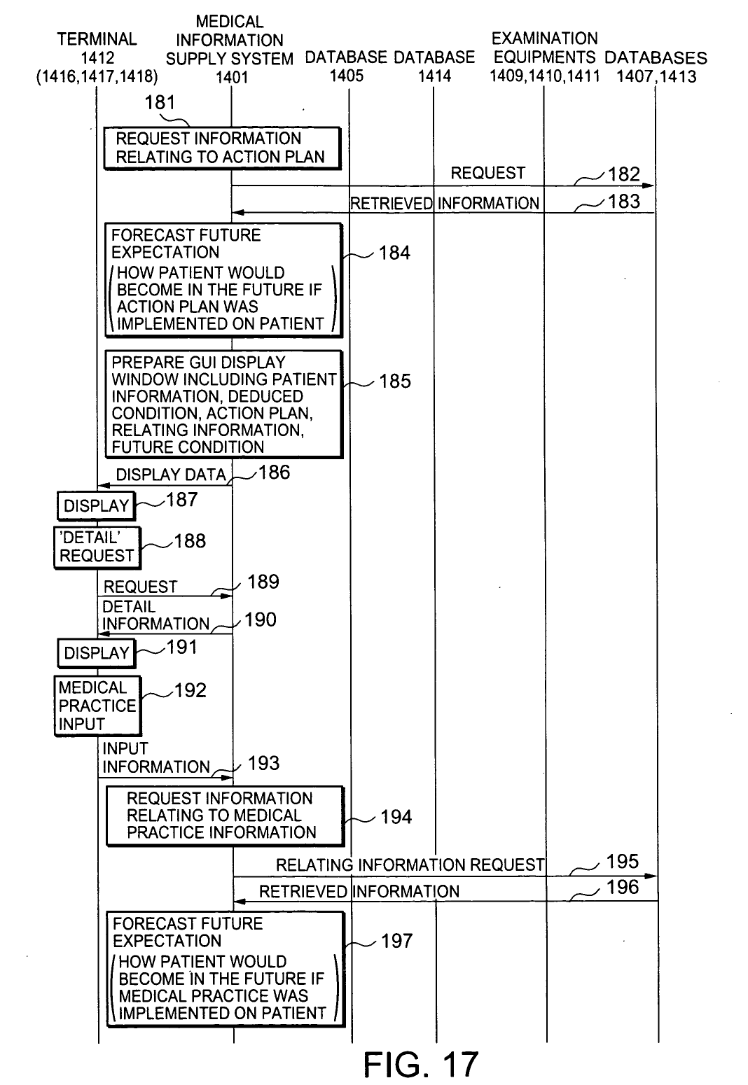

Turning to FIG. 17, the information collection section 1427 requests, for example, the pharmaceutical information database 1407 and the latest medical information database 1413 to supply information relating to the prepared candidate action plans (steps 181 and 182). In response to the request, the pharmaceutical information database 1407 and the latest medical information database 1413 retrieve necessary information from stored information in accordance with, for example, the treatment and a type of medicine to be administered. When necessary information meeting the request is retrieved, the retrieved information regarding medicines and treatment case examples is transmitted to the information collection section 1427 (step 183).

The future condition forecast section 1429 forecasts a future condition of the patient (or a future expectation) under each candidate action plan. In other words, the future condition forecast section 1429 forecasts how the patient would become in the future if at least one of treatments included in each candidate action plan was implemented on the patient. The forecast may be implemented based on information of patient's symptom, sex, age, body build, life style, anamnesis, current hospital record, blood examination result, treatment, a type of medicine to be administered, a dose of the medicine, and a latest medical information. A QOL, an EBM, and mortality are presented as the future expectation of the patient (step 184).

The display information sort-out section 1431 sorts out the information collected by the data collection section 1422, the disease name deduced by the deduction section 1424, the candidate action plans prepared by the plan preparation section 1425, the relating information collected by the information collection section 1427, and the future conditions forecasted by the future condition forecast section 1429, in accordance with the user role information, the situation information, and the access authority given to the user.

After the sort-out, the display window preparation section 1432 adapts the sorted out information to a GUI display window template. Accordingly, a display window as shown in FIG. 9 or 10 may be prepared in a GUI form (step 185). Data of the prepared display window are transmitted to the consultation room terminal 1412 from the communication section 1420 through the network 1402 (step 186). The transmitted data are displayed as the GUI display window in the consultation room terminal 1412 (step 181).

When the user clicks on the ‘Detail’ button for one of the candidate action plans in the GUI display window (step 188), such click-on information is transmitted to the input section 1421 as a detail information request (step 189). In the medical information supply system 1401, the display window preparation section 1432 prepares a detail information display window including detail information relating to the clicked action plan. Data of the prepared detail information display window are transmitted to the consultation room terminal 1412 (step 190). The transmitted data are displayed as the detail information display window in the consultation room terminal 1412 (step 191)

Meanwhile, whether as a result of studying the information supplied from the medical information supply system 1401 or not, the user can input medical practice information from the consultation room terminal 1412 (step 192). The medical practice information may relate to what the user is thinking of actually implementing on the patient, such as, for example, a treatment. Therefore, the medical practice information may alternatively be expressed as medical plan information or hypothetical medical practice information. Such hypothetical medical practice information input by the user is transmitted to the input section 1421 (step 193). In response to reception of the information, the information collection section 1427 requests the pharmaceutical information database 1407 and the latest medical information database 1413 to supply information relating to the received hypothetical medical practice information (steps 194 and 195).

In response to the request, the pharmaceutical information database 1407 and the latest medical information database 1413 retrieve necessary information from stored information in accordance with the hypothetical medical practice information. When necessary information meeting the request is retrieved, the retrieved information is transmitted to the information collection section 1427 (step 196).

The future condition forecast section 1429 forecasts a future condition of the patient (or a future expectation) under the hypothetical medical practice. In other words, the future condition forecast section 1429 forecasts how the patient would become in the future if the hypothetical medical practice was implemented on the patient. The forecast may be implemented in a manner similar to step 184. A QOL, an EBM, and mortality are presented as the future expectation of the patient (step 197).

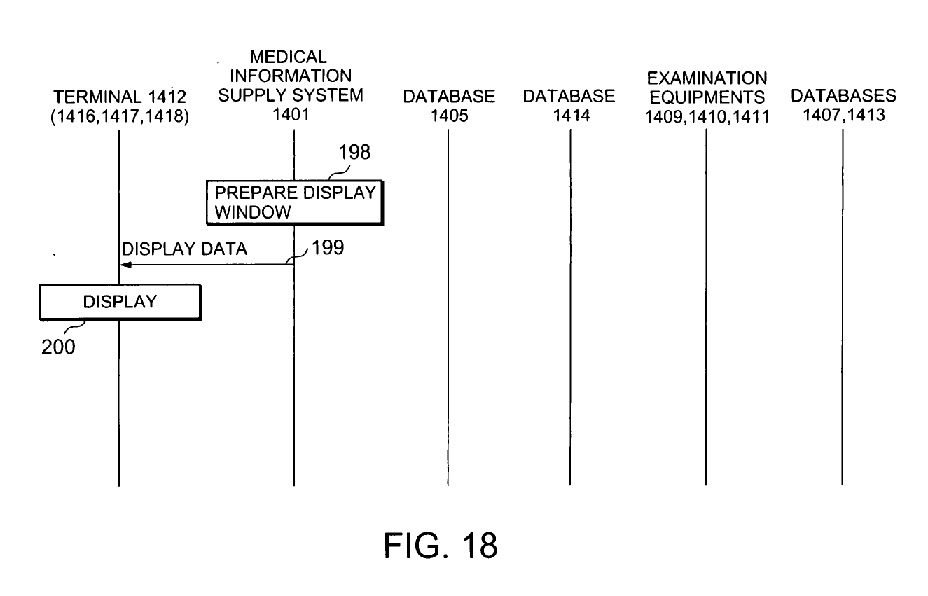

A display window including the forecasted future expectation is prepared by the display window preparation section 1432 (step 198). Data of the prepared display window are transmitted to the consultation room terminal 1412 from the communication section 1420 through the network 1402 as shown in FIG. 18 (step 199). The transmitted data are displayed as the display window in the consultation room terminal 1412 (step 200). Accordingly, the user can obtain the future expectation in case of the hypothetical medical practice.

According to the medical information supply system 1401 in the third embodiment, it may be possible to obtain necessary information of, for example, a health, medicine, and welfare anywhere and anytime a user would like to obtain. Further, since a plurality piece of information can be integrated, it may be possible to increase a value of the information, compared to a single use of the information. A user is supplied only necessary information from vast medical information in accordance with user role information and situation information. Further, the user can be supplied a wide variety of information, such as candidate action plans, detail information of the plans, latest medical information relating to the plan, and future expectation resulting from the plan. Accordingly, the user can perform a preferred action for a patient promptly.

The medical information supply system may make it easier to establish the cyber hospital system by a doctor, a medical specialist, or from a medical examination facility. Further, the medical information supply system can supply doctors such as a primary physician and a medical specialist with valuable information for a diagnosis and a treatment. Therefore, the application of the medical information supply system may effectively improve a value of the cyber hospital system although the medical information supply system is not necessarily required for the cyber hospital system.

The embodiments of the present invention described above are examples described only for making it easier to understand the present invention, and are not described for the limitation of the present invention. Consequently, each component and element disclosed in the embodiments of the present invention may be redesigned or modified to its equivalent within a scope of the present invention. Furthermore, any possible combination of such components and elements may be included in a scope of the present invention as long as an advantage similar to those obtained according to the above disclosure in the embodiments of the present invention is obtained.

Numerous modifications and variations of the present invention are possible in light of the above teachings. It is therefore to be understood that within the scope of the appended claims, the invention may be practiced otherwise than as specifically described herein.

1. A cyber hospital system connected to a remote terminal through a network, the system comprising:
an input unit configured to input patient condition information from the remote terminal through the network;
a first processor configured to collect first doctor information based on the patient condition information; and
a transmitter configured to transmit the first doctor information to the remote terminal.

2. The system according to claim 1, further comprising a database configured to store the first doctor information.

3. The system according to claim 1, wherein the system is connected to a database storing at least the first doctor information, and wherein the first processor collects the first doctor information from the database.

4. The system according to claim 3, wherein the database is provided at an external location.

5. The system according to claim 1, wherein a patient's primary physician is determined based on the first doctor information.

6. The according to claim 1, wherein a medical specialist for the patient is determined based on the first doctor information.

7. The system according to claim 1, wherein the input unit is configured to input patient identification information from the remote terminal.

8. The system according to claim 7, further comprising a second processor configured to collect patient information based on the patient identification information, and wherein the first processor collects the first doctor information based on the patient condition information and the patient information.

9. The system according to claim 8, further comprising a database configured to store the patient information.

10. The system according to claim 8, wherein the system is connected to a database storing at least the patient information, and wherein the second processor collects the patient information from the database.

11. The system according to claim 10, wherein the database is provided at an external location.

12. The system according to claim 8, wherein the transmitter is configured to transmit the patient condition information and the patient information to a first doctor determined based on the first doctor information by the remote terminal.

13. The system according to claim 1, wherein the remote terminal is used by the patient.

14. The system according to claim 1, wherein the remote terminal is used in an ambulance.

15. The system according to claim 1, wherein the remote terminal is used in a hospital.

16. The system according to claim 1, wherein a first doctor is determined based on the first doctor information by the remote terminal.

17. The system according to claim 16, further comprising a third processor configured to establish a connection between the remote terminal and a second remote terminal used by the first doctor.

18. The system according to claim 16, wherein the first processor is further configured to collect second doctor information based on the patient condition information, and wherein the transmitter is configured to transmit the second doctor information to the remote terminal and a second remote terminal used by the first doctor.

19. The system according to claim 18, further comprising a database configured to store the second doctor information.

20. The system according to claim 18, wherein the system is connected to a database storing at least the second doctor information, wherein the first processor collects the second doctor information from the database.

21. The system according to claim 20, wherein the database is provided at an external location.

22. The system according to claim 18, wherein the input unit is configured to input patient identification information from the remote terminal.

23. The system according to claim 22, further comprising a second processor configured to collect patient information based on the patient identification information, wherein the first processor collects the second doctor information based on the patient condition information and the patient information.

24. The system according to claim 23 wherein the transmitter is configured to transmit the patient condition information and the patient information to a second doctor determined based on the second doctor information.

25. The system according to claim 24, wherein the second doctor is a medical specialist.

26. The system according to claim 24, wherein the second doctor is an interpretation doctor.

27. The system according to claim 1, wherein the first processor is configured to collect medical facility information based on the patient condition information, and wherein the transmitter is configured to transmit the medical facility information to the remote terminal and a second remote terminal used by a first doctor determined based on the first doctor information by the remote terminal.

28. The system according to claim 27, further comprising a database configured to store the medical facility information.

29. The system according to claim 27, wherein the system is connected to a database storing at least the medical facility information, wherein the processor collects the medical facility information from the database.

30. The system according to claim 29, wherein the database is provided at an external location.

31. The system according to claim 27, wherein the input unit is further configured to input patient identification information from the remote terminal.

32. The system according to claim 31, further comprising a second processor configured to collect patient information based on the patient identification information, and wherein the first processor collects the medical facility information based on the patient condition information and the patient information.

33. The system according to claim 32, wherein the transmitter is further configured to transmit the patient condition information and the patient information to a medical facility determined based on the medical facility information.

34. The system according to claim 1, further comprising a second processor configured to collect medical information based on the patient condition information, and wherein the transmitter is further configured to transmit the medical information to the remote terminal and a second remote terminal used by a first doctor determined based on the first doctor information by the remote terminal.

35. The system according to claim 34, further comprising a database configured to store the medical information.

36. The system according to claim 34, wherein the system is connected to a database storing at least the medical information, wherein the second processor collects the medical information from the database.

37. The system according to claim 36, wherein the database is provided at an external location.

38. The system according to claim 34, wherein the input unit is further configured to input patient identification information from the remote terminal.

39. The system according to claim 38, wherein the second processor is configured to collect patient information based on the patient identification information, and wherein the second processor collects the medical information based on the patient condition information and the patient information.

40. The system according to claim 24, further comprising a third processor configured to establish a connection between the second remote terminal and a third remote terminal used by the second doctor.

41. The system according to claim 24, further comprising a third processor configured to establish a connection between a third remote terminal used by the second doctor and a medical facility.

42. The system according to claim 33, further comprising a third processor configured to establish a connection between the second remote terminal and the medical facility.

43. A cyber hospital system connected to a remote terminal through a network, the system comprising:
an input unit configured to input patient location information from the remote terminal through the network;
a processor configured to collect medical facility information based on the patient location information; and
a transmitter configured to transmit the medical facility information to the remote terminal.

44. A medical information supply system connected to a remote terminal and a plurality of databases, the system comprising:
an input unit configured to input first patient information and use information from the remote terminal;
a processor configured to make a request to one or more of the databases so as to collect second patient information based on the first patient information and the use information; and
a transmitter configured to transmit the second patient information to the remote terminal.

45. The system according to claim 44, wherein the first patient information includes patient identification information and patient health condition information.

46. The system according to claim 44, wherein the use information includes user identification information, user role information, and situation information.

47. The system according to claim 46, wherein the situation information includes user location information.

48. The system according to claim 44, wherein the processor specifies an information type for the request and makes the request based on the information type.

49. The system according to claim 44, further comprising a deduction unit configured to deduce a medical condition of the patient based on the first and second patient information.

50. The system according to claim 49, wherein the deduced medical condition represent a disease name.

51. The system according to claim 49, further comprising a first preparation unit configured to prepare a medical action plan based on the deduced medical condition.

52. The system according to claim 51, further comprising a forecast unit configured to forecast a future condition of the patient which is expected by implementing the medical action plan on the patient.

53. The system according to claim 52, further comprising a second preparation unit configured to sort out display information from the deduced medical condition, the prepared medical action plan, and the forecast future condition and to prepare display data including the sorted out display information, wherein the display data are transmitted to the remote terminal as a part of the second patient information.

54. The system according to claim 52, further comprising a second preparation unit configured to prepare display data including the deduced medical condition, the prepared medical action plan, and the forecast future condition, wherein the display data are transmitted to the remote terminal as a part of the second patient information.

55. The system according to claim 51, further comprising a second processor configured to make a request to one or more of the databases so as to collect relating information which relates to the medical action plan, wherein, when detail information of the medical action plan is requested by the remote terminal, the relating information is transmitted to the remote terminal.

56. The system according to claim 44, further comprising a forecast unit configured to forecast a future condition of the patient which is expected by implementing a medical practice represented in medical practice information on the patient when the input unit is further configured to input the medical practice information from the remote terminal.

57. The system according to claim 56, wherein the transmitter is further configured to transmit the forecasted future condition to the remote terminal.

58. The system according to claim 44, further comprising a preparation unit configured to prepare a display window to be displayed in the remote terminal, the display window including a virtual patient body, wherein the second patient information relating to a part designated on the virtual patient body is displayed in the display window.

59. The system according to claim 58, further comprising a deduction unit configured to deduce a disease name of the patient based on the first and second patient information, wherein the virtual patient body is marked where the deduced disease name is related.

60. The system according to claim 44, further comprising a second processor configured to request the remote terminal to input additional information when the additional missing is determined to be missing.

61. The system according to claim 7, further comprising a second processor configured to collect patient information based on the patient identification information, to deduce a medical condition of the patient based on the first and patient information, to prepare a medical action plan based on the deduced medical condition, and to forecast a future condition of the patient which is expected by implementing the medical action plan on the patient.

62. The system according to claim 61, wherein the transmitter is further configured to transmit at least one of the patient condition information, the patient information, the deduced medical condition, the prepared medical action plan, and the forecasted future condition to a first doctor determined based on the first doctor information by the remote terminal.

63. A method of supplying a remote terminal with certain information, the method comprising steps of:
inputting first patient information and use information from the remote terminal;
making a request to one or more databases so as to collect second patient information based on the first patient information and the use information; and
transmitting the second patient information to the remote terminal as the certain information.

64. A method of medical information processing, the method comprising steps of:
inputting patient condition information and patient identification information from a remote terminal through a network;
collecting patient information based on the patient identification information;
deducing a medical condition of the patient based on the patient condition information and the patient information;
preparing a medical action plan based on the deduced medical condition; and
forecasting a future condition of the patient which is expected by implementing the medical action plan on the patient.
